(19) United States
(12) Patent Application Publication
(10) Pub. No.: US 2026/0260740 A1
(43) Pub. Date: Sep. 3, 2026

(54) PRIORITIZATION OF HEALTHCARE RESOURCES IN CANCER CARE

(71) Applicant: Harbinger Health, Inc., Cambridge, MA (US)

(72) Inventors: Anthony P. Shuber, Whitinsville, MA (US); Miguel Williams, Roslindale, MA (US); Ursula Winter, Cambridge, MA (US)

(21) Appl. No.: 19/573,525

(22) Filed: Mar. 20, 2026

Related U.S. Application Data
(63) Continuation of application No. PCT/US2026/017085, filed on Feb. 27, 2026.
(60) Provisional application No. 63/765,222, filed on Feb. 28, 2025.
(60) Provisional application No. 63/775,072, filed on Mar. 20, 2025.
(60) Provisional application No. 63/852,392, filed on Jul. 28, 2025.
(60) Provisional application No. 63/853,262, filed on Jul. 29, 2025.
(60) Provisional application No. 63/902,807, filed on Oct. 21, 2025.
(60) Provisional application No. 63/910,448, filed on Nov. 3, 2025.

(51) Int. Cl. G16H 40/20; G16B 25/20; G16B 40/00; G16H 50/20; G16H 50/30; G16H 50/70

(52) U.S. Cl. CPC G16H 40/20; G16B 25/20; G16B 40/00; G16H 50/20; G16H 50/30; G16H 50/70

(57) ABSTRACT

Methods involve analyzing data of at least one analyte from one or more samples obtained from a subject, categorizing the subject into a risk category according to the analysis of the analyte data, and assigning a frequency of healthcare utilization for the subject according to the categorized risk category for the subject. Subjects that are identified to be at higher risk for cancer can be assigned a higher frequency of healthcare utilization whereas subjects identified to be at a lower risk for cancer can be assigned a lower frequency of healthcare utilization. Given the limitations of healthcare resources, this enables the prioritization of longitudinal tracking and follow up visits for subjects that are at highest risk.

### CROSS-REFERENCE TO RELATED APPLICATIONS

This application is a Continuation of International Application No. PCT/US2026/017085 filed Feb. 27, 2026, which claims the benefit of and priority to U.S. Provisional Patent Application No. 63/765,222 filed Feb. 28, 2025, U.S. Provisional Patent Application No. 63/775,072 filed Mar. 20, 2025, U.S. Provisional Patent Application No. 63/852,392 filed Jul. 28, 2025, U.S. Provisional Patent Application No. 63/853,262 filed Jul. 29, 2025, U.S. Provisional Patent Application No. 63/902,807 filed Oct. 21, 2025, and U.S. Provisional Patent Application No. 63/910,448 filed Nov. 3, 2025, the entire disclosure of each of which is hereby incorporated by reference in its entirety for all purposes.

### BACKGROUND

Recent expansions in cancer screening guidelines across multiple cancer types have added substantial numbers of patients to screening populations, dramatically increasing the volume of patients undergoing non-invasive screening tests. This expanded screening population has created a critical resource allocation problem: the healthcare system's diagnostic capacity is limited by specialist availability, diagnostic procedure time slots, and support staff, and is increasingly overwhelmed by the volume of screening-positive patients requiring follow-up evaluation. Current clinical practice employs a binary approach where any positive screening test triggers automatic referral regardless of individual risk level (e.g. a “yes or no”, often with a high false positive rate), resulting in inefficient allocation of scarce resources and potential delays for genuinely high-risk patients who require urgent evaluation. Existing computer-based diagnostic systems focus on determining cancer presence/absence or generating treatment recommendations, requiring substantial computational resources to process complex analyses, but fail to address the specific technical challenge of the post-screening, pre-diagnostic procedure context. What is needed is an efficient methodology specifically designed to rapidly categorize patients into clinically actionable risk tiers, thereby optimizing limited colonoscopy resources across the expanded screening population without attempting to render diagnostic conclusions or therapeutic recommendations.

### SUMMARY

Disclosed herein are methods for prioritizing healthcare resources in cancer care. In particular embodiments, methods disclosed herein involve analyzing data of at least one analyte from a sample obtained from a subject, categorizing the subject into a risk category according to the analysis of the analyte data, and assigning a frequency of healthcare utilization for the subject according to the categorized risk category for the subject (e.g., high, medium or low risk). Therefore, subjects who are categorized in a high risk category can be prioritized over subjects who are categorized in less risky categories. For example, subjects categorized in a high risk category undergo more frequent follow up examinations in comparison to subjects categorized in a less risky category. This ensures that efforts in the early detection of cancer can continue while appropriately allocating healthcare resources to subjects that most need it.

The methods and systems disclosed herein provide a concrete technological solution to the specific problem of efficiently allocating limited healthcare resources in the face of increasing demand for colonoscopy procedures following expanded colorectal cancer screening guidelines. The disclosed methods employ a particular technical implementation involving analysis of specific biomarkers to stratify patients into clinically actionable risk categories. As a particular example, methods can involve implementation of qMSP to analyze methylation statuses to stratify patients. This technological approach achieves superior resource optimization and speed. Altogether, the disclosed methods represent an unconventional approach to patient care using methodologies that produce measurable real-world improvements: faster turnaround times enabling timely clinical decision-making, accessibility without requiring expensive infrastructure, and concrete healthcare utilization frequency assignments that optimize the use of scarce resources across expanded screening populations.

Methods disclosed herein additionally improve a computer's ability to process and integrate complex multi-dimensional biological data (including biomarker levels, patient history, and test results) through an algorithmic framework specifically designed to generate stratified risk categories for colorectal cancer follow-up. This technological solution addresses the computer-centric problem of efficiently processing heterogeneous medical data to produce clinically actionable risk tiers without necessarily rendering a diagnostic conclusion or treatment recommendation.

Disclosed herein is a method for prioritizing healthcare resources for patient care, the method comprising; obtaining sequencing data of cfDNA from a quantitative methylation-specific polymerase chain reaction (qMSP) assay or next generation sequencing assay, the cfDNA obtained from one or more samples obtained from a subject, wherein the subject was previously identified as at risk of a cancer through a prior screen; stratifying the subject into a risk category by analyzing the qMSP data of the cfDNA from the one or more samples obtained from the subject; and assigning the subject a category of healthcare utilization according to the stratified risk category of the subject.

In various embodiments, methods further comprise obtaining data of one or more additional analytes and wherein the stratification of the subject further comprises analyzing the obtained data of the one or more additional analytes. In various embodiments, the one or more additional analytes comprises a protein analyte and/or a nucleic acid analyte. In various embodiments, the one or more additional analytes comprises a protein analyte, a DNA analyte, and a RNA analyte. In various embodiments, the risk category is one of a high risk, intermediate risk, or low risk. In various embodiments, a frequency of healthcare utilization determined for the subject in the high risk category is greater than the frequency of healthcare utilization determined for a subject in the intermediate risk category and/or the low risk category. In various embodiments, the data of the one or more additional analytes further comprises data of one or more lipids or data from one or more metabolites. In various embodiments, obtaining data of one or more additional analytes comprises performing a protein assay or a metabolite detection assay. In various embodiments, the data of one or more additional analytes comprises nucleic acid data, wherein the nucleic acid data is obtained by performing sequencing of nucleic acids derived from exosomes, sequencing of nucleic acids derived from DNA, or sequencing of nucleic acids derived from RNA. In various embodiments, performing sequencing comprises sequencing converted nucleic acids derived from cell-free DNA (cfDNA).

In various embodiments, the converted nucleic acids comprise bisulfite converted nucleic acids derived from cfDNA. In various embodiments, performing the sequencing assay comprises performing hybrid capture and/or polymerase chain reaction (PCR), optionally wherein the PCR is a methylation-specific PCR. In various embodiments, the protein assay comprises a protein immunoassay, an enzyme linked immunosorbent assay (ELISA), radioimmunoassay (RIA), mass spectrometry (MS), or an antibody-based proximity binding assay. In various embodiments, the metabolite detection assay comprises nuclear magnetic resonance (NMR), mass spectrometry (MS), or Infrared spectroscopy (IS). In various embodiments, the qMSP data of cfDNA comprises quantitative methylation statuses of a plurality of genomic sites. In various embodiments, the plurality of genomic sites comprises one or more genomic regions shown in any of Tables 4-9. In various embodiments, the plurality of genomic sites comprises one or more genomic regions shown in any of Tables 8 or 9.

In various embodiments, methods further comprise obtaining imaging data of the subject, and wherein stratifying the subject into a risk category further comprises analyzing the image data of the subject. In various embodiments, the image data comprises one or more images of a tissue or of a cell of the subject. In various embodiments, the healthcare utilization comprises a medical procedure, a future visit to a hospital, the use of a specialist, a diagnostic testing, a diagnostic imaging, a biopsy, a medical appointment, and/or a prophylactic medication. In various embodiments, the medical procedure comprises a colonoscopy. In various embodiments, diagnostic imaging comprises one or more of an ultrasound scan, a MRI scan, or a CT scan. In various embodiments, the prior screen comprises a family history evaluation, a life style evaluation, an age group, a demographic group, and/or a prior cancer diagnostic test. In various embodiments, the prior screen comprises COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, imaware Prostate Cancer screening test a PSA blood test, a mammogram, a colonoscopy, a Pap smear, am HPV test, a biopsy, a hereditary cancer screen (for example the Natera Empower test, Guardant Hereditary cancer test, Myriad Genetics hereditary cancer test or Labcorp hereditary cancer test), low-dose computed tomography (LDCT), a prenuvo cancer scan, or the Grail Galleri multi-cancer screening.

In various embodiments, cancer is any of gastrointestinal (GI) cancer, acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In various embodiments, the GI cancer is colorectal cancer, esophageal cancer, stomach cancer, gallbladder cancer, liver cancer, pancreatic cancer, small intestinal risk, rectal cancer, appendix cancer, anal cancer, and/or colon cancer. In various embodiments, methods further comprise in response to not assigning the subject to a high risk category, performing longitudinal tracking of the subject over one or more additional timepoints. In various embodiments, the one or more additional timepoints comprises between 1 and 50 additional time points. In various embodiments, performing longitudinal tracking of the subject over one or more additional timepoints comprises repeating steps (a)-(c) at each additional timepoint. In various embodiments, the subject has a first-degree relative who was previously diagnosed with cancer prior to a threshold age. In various embodiments, the threshold age is 45 years old, 50 years old, or 55 years old.

Additionally disclosed herein is a method for prioritizing healthcare resources for patient care, the method comprising; for each of a plurality of subjects previously identified as at risk of a cancer through a prior screen; obtaining sequencing data of cfDNA from a quantitative methylation-specific polymerase chain reaction (qMSP) assay or next generation sequencing assay, the cfDNA obtained from one or more samples obtained from the subject; stratifying the subject into a risk category by analyzing the qMSP data of the cfDNA from the one or more samples obtained from the subject; and assigning the subject a category of healthcare utilization according to the stratified risk category of the subject, wherein different subjects of the plurality of subjects are assigned to different frequencies of healthcare utilization.

In various embodiments, methods further comprise for each of a plurality of subjects, obtaining data of one or more additional analytes and wherein the stratification of the subject further comprises analyzing the obtained data of the one or more additional analytes. In various embodiments, the one or more additional analytes comprises a protein analyte and/or a nucleic acid analyte. In various embodiments, the one or more additional analytes comprises a protein analyte, a DNA analyte, and a RNA analyte. In various embodiments, the risk category is one of a high risk, intermediate risk, or low risk. In various embodiments, a frequency of healthcare utilization determined for a subject in the high risk category is greater than a frequency of healthcare utilization determined for a subject in the intermediate risk category and/or the low risk category. In various embodiments, the data of the one or more additional analytes further comprises data of one or more lipids or data from one or more metabolites. In various embodiments, obtaining data of one or more additional analytes comprises performing a protein assay or a metabolite detection assay.

In various embodiments, the data of one or more additional analytes comprises nucleic acid data, wherein the nucleic acid data is obtained by performing sequencing of nucleic acids derived from exosomes, sequencing of nucleic acids derived from DNA, or sequencing of nucleic acids derived from RNA. In various embodiments, performing sequencing comprises sequencing converted nucleic acids derived from cell-free DNA (cfDNA). In various embodiments, the converted nucleic acids comprise bisulfite converted nucleic acids derived from cfDNA. In various embodiments, performing the sequencing assay comprises performing hybrid capture and/or polymerase chain reaction (PCR), optionally wherein the PCR is a methylation-specific PCR. In various embodiments, the protein assay comprises a protein immunoassay, an enzyme linked immunosorbent assay (ELISA), radioimmunoassay (RIA), mass spectrometry (MS), or an antibody-based proximity binding assay. In various embodiments, the metabolite detection assay comprises nuclear magnetic resonance (NMR), mass spectrometry (MS), or Infrared spectroscopy (IS). In various embodiments, the qMSP data of cfDNA comprises quantitative methylation statuses of a plurality of genomic sites. In various embodiments, the plurality of genomic sites comprises one or more genomic regions shown in any of Tables 4-9. In various embodiments, the plurality of genomic sites comprises one or more genomic regions shown in any of Tables 8 or 9.

In various embodiments, methods further comprise obtaining imaging data of the subject, and wherein stratifying the subject into a risk category further comprises analyzing the image data of the subject. In various embodiments, the image data comprises one or more images of a tissue or of a cell of the subject. In various embodiments, the healthcare utilization comprises a medical procedure, a future visit to a hospital, the use of a specialist, a diagnostic testing, a diagnostic imaging, a biopsy, a medical appointment, and/or a prophylactic medication. In various embodiments, the medical procedure comprises a colonoscopy. In various embodiments, diagnostic imaging comprises one or more of an ultrasound scan, a MRI scan, or a CT scan. In various embodiments, the prior screen comprises a family history evaluation, a life style evaluation, an age group, a demographic group, and/or a prior cancer diagnostic test. In various embodiments, the prior screen comprises COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, imaware Prostate Cancer screening test a PSA blood test, a mammogram, a colonoscopy, a Pap smear, am HPV test, a biopsy, a hereditary cancer screen (for example the Natera Empower test, Guardant Hereditary cancer test, Myriad Genetics hereditary cancer test or Labcorp hereditary cancer test), low-dose computed tomography (LDCT), a prenuvo cancer scan, or the Grail Galleri multi-cancer screening. In various embodiments, cancer is any of gastrointestinal (GI) cancer, acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In various embodiments, the GI cancer is colorectal cancer, esophageal cancer, stomach cancer, gallbladder cancer, liver cancer, pancreatic cancer, small intestinal risk, rectal cancer, appendix cancer, anal cancer, and/or colon cancer.

In various embodiments, methods further comprise for each subject of the plurality of subjects that are not assigning to a high risk category, performing longitudinal tracking of the subject over one or more additional timepoints. In various embodiments, the one or more additional timepoints comprises between 1 and 50 additional time points. In various embodiments, performing longitudinal tracking of the subject over one or more additional timepoints comprises repeating steps (a)-(c) at each additional timepoint. In various embodiments, one or more of the subjects of the plurality of subjects has a first-degree relative who was previously diagnosed with cancer prior to a threshold age. In various embodiments, the threshold age is 45 years old, 50 years old, or 55 years old.

Additionally disclosed herein is a non-transitory computer readable medium comprising instructions that, when executed by a processor, cause the processor to perform methods disclosed herein. Additionally disclosed herein is a computer system comprising: a processor; and a non-transitory computer readable medium comprising instructions that, when executed by a processor, cause the processor to perform the methods disclosed herein.

### BRIEF DESCRIPTION OF THE DRAWINGS

These and other features, aspects, and advantages of the present invention will become better understood with regard to the following description and accompanying drawings. It is noted that wherever practicable, similar or like reference numbers may be used in the figures and may indicate similar or like functionality. For example, a letter after a reference numeral, such as “sample 115A,” indicates that the text refers specifically to the element having that particular reference numeral. A reference numeral in the text without a following letter, such as “sample 115,” refers to any or all of the elements in the figures bearing that reference numeral (e.g. “sample 115” in the text refers to reference numerals “sample 115A” and/or “sample 115B” in the figures).

Figure (FIG. 1A depicts an overall flow diagram for prioritizing healthcare resources in cancer care, in accordance with a first embodiment.

FIG. 1B depicts an overall flow diagram for prioritizing healthcare resources in cancer care, in accordance with a second embodiment.

FIG. 2A shows an example flow diagram for analyzing multianalyte information, in accordance with an embodiment.

FIG. 2B shows canonical positive predictive value (PPV) thresholding, in accordance with an embodiment.

FIG. 2C shows an example patient stratification based on predictive value scaling, in accordance with an embodiment.

FIG. 2D shows an example patient stratification based on predictive value binning, in accordance with an embodiment.

FIG. 3A shows an example flow process for prioritizing healthcare resources in cancer care, in accordance with an embodiment.

FIG. 3B shows an example flow process for processing a sample to generate data for an analyte, in accordance with an embodiment.

FIG. 3C shows an example flow process for generating a cancer prediction for a subject, in accordance with an embodiment.

FIG. 3D shows an example flow process for prioritizing healthcare resources by analyzing qMSP sequencing data of a high-risk subject, in accordance with an embodiment.

FIG. 4 illustrates an example computer for implementing the entities shown in FIGS. 1A-1B, 2, and 3.

FIG. 5 depicts incidence and death rates of common cancers as well as improved survival of subjects for those common cancers depending on when the cancer was diagnosed.

FIG. 6 depicts the burden on healthcare utilization, specifically colonoscopies, for early detection of cancer.

FIG. 7 depicts the value of effective patient stratification which results in reduced costs and increased lives saved.

FIG. 8 is a diagrammatic depiction of stratification of subjects which enables optimized allocation of healthcare resources.

FIG. 9 depicts the risk stratification step that is performed either between Tier 1 and Tier 2, or during tier 3 monitoring stages.

FIG. 10 depicts an example analysis involving a multi-omic approach (e.g., including analysis of cfDNA, lipidomics, exosomal RNA, metabolomics, and/or proteins from circulating tumor cells (CTCs).

FIG. 11 depicts the additive benefit of analyzing DNA and protein (in addition to RNA).

FIGS. 12A-12C depict exemplary methodologies for analyzing multi-analyte samples.

FIGS. 13A-13E depict exemplary multianalytes in liquid biopsies.

FIG. 14 shows a heatmap of methylation for LBR markers in non-cancer cfDNA and cancer tissue.

FIG. 15 shows a heatmap of methylation for SIMBA markers in non-cancer cfDNA and cancer tissue.

FIG. 16 shows a UMAP of SIMBA biomarkers and cancer tissue samples.

FIG. 17 shows ROC curve comparison of LBR and SIMBA biomarker sets.

FIG. 18 shows sensitivity by cancer type at 80% target specificity using LBR biomarkers.

FIG. 19 shows sensitivity by cancer type at 80% target specificity using LBR biomarkers.

FIG. 20 shows sensitivity by cancer stage at 80% target specificity using LBR biomarkers.

FIGS. 21A-21B show CT scores in 10 Cancer and 10 Non-Cancer Patients using qMSP biomarkers (BCAT1, SDC2, and SEPT9).

### DETAILED DESCRIPTION

### Definitions

Terms used in the claims and specification are defined as set forth below unless otherwise specified.

The terms “subject,” “patient,” and “individual” are used interchangeably and encompass a cell, tissue, or organism, human or non-human, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

It must be noted that, as used in the specification, the singular forms “a,” “an” and “the” include plural referents unless the context clearly dictates otherwise.

### Overview

Generally, methods disclosed herein involve analyzing data of one or more analytes from one or more samples obtained from a subject, categorizing the subject into a risk category according to the analysis of the data of the one or more analytes, and assigning a frequency of healthcare utilization for the subject according to the categorized risk category for the subject. In various embodiments, the subject may be an individual not previously diagnosed with a cancer (e.g., the subject may be considered to be healthy). In some embodiments, the subject may be exhibiting symptoms indicative of cancer. For example, a subject suspected of having colon cancer may be displaying persistent changes in bowel habits, blood in or on the stool, persistent abdominal discomfort, unexplained weight loss and fatigue. The subject may be following current guidelines for monitoring of cancer (e.g., adults begin screening for colorectal cancer at the age of 45). Therefore, subjects that are identified to be at higher risk for cancer can be assigned a higher frequency of healthcare utilization whereas subjects identified to be at a lower risk for cancer can be assigned a lower frequency of healthcare utilization. Given the limitations of healthcare resources, this enables the prioritization of longitudinal tracking and follow up visits for subjects that are at highest risk.

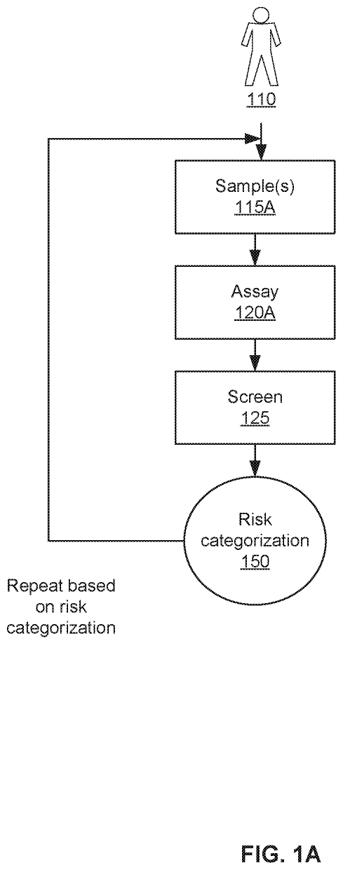

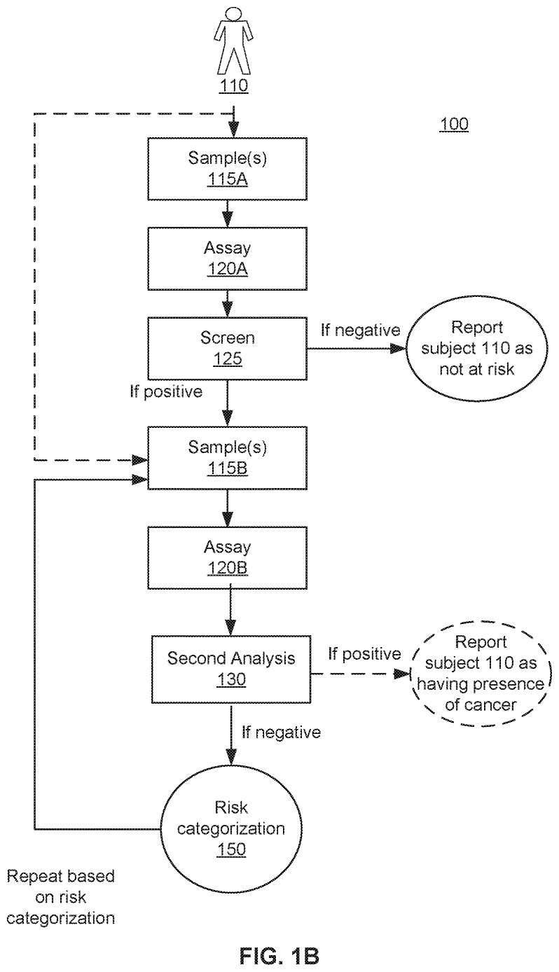

In various embodiments, the methods disclosed herein involve a single analysis in which the single analysis involves analyzing data of multiple analytes from one or more samples obtained from a subject and categorizing the subject into a risk category according to the analysis of the multiple analyte data, and assigning a frequency of healthcare utilization for the subject according to the categorized risk category for the subject. Embodiments of the single tier analysis are shown in FIG. 1A, which depicts an overall flow diagram for prioritizing healthcare resources in cancer care, in accordance with a first embodiment.

In various embodiments, the methods disclosed herein involve a multi-tier analysis involving at least a first analysis (referred to herein as a Tier 1 analysis) followed by a second analysis (referred to herein as a Tier 2 analysis). As discussed in further detail herein, the Tier 1 analysis may involve a screen and can be a rapid, cost-effective test. Thus, the Tier 1 analysis can be suitable for screening large numbers of subjects to eliminate subjects that are not at risk for cancer. The Tier 2 analysis may involve a more complex and/or more expensive test and therefore can be suitable for analyzing subjects that were not eliminated following the Tier 1 analysis. Embodiments of the multitier analysis are shown in FIG. 1B, which depicts an overall flow diagram for prioritizing healthcare resources in cancer care, in accordance with a second embodiment.

In various embodiments, the Tier 1 analysis may involve a cancer test, such as a commercially available cancer test, examples of which include any of COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, imaware Prostate Cancer screening test,, a PSA blood test, a mammogram, a colonoscopy, a Pap smear, am HPV test, a biopsy, a hereditary cancer screen (for example the Natera Empower test, Guardant Hereditary cancer test, Myriad Genetics hereditary cancer test or Labcorp hereditary cancer test), low-dose computed tomography (LDCT), a prenuvo cancer scan, or the Grail Galleri multi-cancer screening. The Tier 1 analysis may involve identifying, through the cancer test, that the subject is at risk for cancer. In various embodiments, the Tier 2 analysis can be performed to validate or contradict the results of the Tier 1 analysis. Thus, this enables more accurate identification and stratification of subjects who would benefit from a particular frequency of healthcare utilization. As an example, the Tier 1 analysis for a subject may involve a cancer test (e.g., COLOGUARD®), which determines that the subject is at risk for cancer. Then, a Tier 2 analysis can be performed for the subject to determine whether the determination of the Tier 1 analysis is correct. In some circumstances, the Tier 2 analysis can determine that the Tier 1 analysis of cancer risk is correct and that the subject is at risk for cancer. In some scenarios, the Tier 2 analysis can determine that the subject is not at risk for cancer, thereby contradicting the conclusion of the Tier 1 analysis. The subject not at risk for cancer can be categorized into a category corresponding to a particular frequency of healthcare utilization (e.g., a high, medium, or low risk that dictates the recommended frequency of the healthcare utilization).

In particular embodiments, the Tier 1 analysis, Tier 2 analysis, and stratification are useful for analyzing subjects of a patient population. For example, the Tier 1 analysis, Tier 2 analysis, and stratification can be used to categorize subjects of a patient population with an incidence rate below a certain threshold percentage. In various embodiments, the threshold percentage is 70%, 60%, 50%, 40%, 30%, 20%, or 10%. In particular embodiments, the threshold percentage is 50%. This is valuable for stratifying a majority of subjects the patient population who are not cancerous, but may have different risks of developing cancer.

FIGS. 1A and 1B introduce a subject 110. Although FIGS. 1A and 1B show the flow process in relation to a single subject 110, in various embodiments, the flow process can be performed for more than a single subject 110 (e.g., for thousands, millions, tens of millions, or hundreds of millions of individuals).

As shown in FIG. 1A, a sample 115A is obtained from the subject 110. As shown in FIG. 1B, multiple samples (e.g., initial sample 115A and/or sample 115B) are obtained from the subject 110. In various embodiments, each sample 115 is any of a blood sample, a stool sample, a urine sample, a mucous sample, or a saliva sample. In particular embodiments, each sample 115 obtained from the subject 110 is a blood sample. The sample 115 can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art. In various embodiments, the one or more samples can be obtained from the subject 110 by a reference lab.

As discussed herein, methods involve assigning a frequency of healthcare utilization for subjects. As used herein, “healthcare utilization” refers to use of resources in a clinical setting. Examples of healthcare utilization can be a medical procedure, such as a surgical procedure, a future visit to a hospital, the use of a specialist, a diagnostic testing, a diagnostic imaging, a biopsy, a medical appointment, and/or a prophylactic medication. In particular embodiments, healthcare utilization refers to a medical procedure, such as a colonoscopy. In particular embodiments, healthcare utilization refers to a fecal immunochemical test. In particular embodiments, healthcare utilization refers to a diagnostic imaging, examples of which include an ultrasound scan, a MRI scan, or a CT scan,

In various embodiments, subjects are determined to be negative for cancer (e.g., no presence of cancer in the subjects). In various embodiments, subjects can be determined to be negative for cancer, but also determined to be at high risk for cancer. Therefore, such subjects can undergo tracking and/or follow up analysis at a shorter frequency than other subjects who are determined to be negative for cancer and determined to be at lower risk for cancer. In various embodiments, the follow up analysis involves repeating the process shown in FIG. 1A and/or FIG. 1B. Therefore, in various embodiments, a plurality of samples are obtained from the subject 110 at a plurality of different points in time. The plurality of different points in time can be dependent on the assigned frequency of healthcare utilization. For example, as shown in FIG. 1A, a sample (e.g., initial sample 115A) can be obtained at a first timepoint. Then, following risk categorization 150, the process is repeated and a new sample 115A is obtained from the subject 110 at a second timepoint. In various embodiments, the initial sample 115A and/or the new sample are each liquid biopsy samples. As shown in FIG. 1B, a first sample 115A can be obtained to perform the screen 125 and a second sample 115B can be obtained to perform the monitoring 128 involving the second analysis 130. Then, following risk categorization 150, the process is repeated and new sample 115B is obtained from the subject 110 at a second timepoint.

In various embodiments, a sample obtained from the subject can be a multianalyte sample. As used herein, a multianalyte sample refers to a sample that includes two or more analytes. Analytes include various biomarkers, examples of which include proteins, metabolites, lipids, and/or nucleic acids (e.g., DNA such as cell-free DNA or RNA). In various embodiments, the multianalyte sample includes protein biomarkers (e.g., protein biomarkers from circulating tumor cells). In various embodiments, the multianalyte sample includes DNA, such as cell-free DNA. In various embodiments, the cell-free DNA can be analyzed to identify methylation profiles, single nucleotide variations (SNVs), and/or copy number variations (CNVs). In particular embodiments, the cfDNA include genomic sequences corresponding to CpG islands for which methylation states are informative of the cancer. In various embodiments, the liquid biopsy sample includes one or more lipids. In various embodiments, the liquid biopsy sample includes extracellular vesicles (EVs). EVs can be analyzed to determine RNA profiles, such as presence or absence of fusion transcripts. In particular embodiments, the multianalyte sample includes protein biomarkers and one or more nucleic acid analytes (e.g., DNA such as cell-free DNA or RNA). In particular embodiments, the multianalyte sample includes protein biomarkers and cell-free DNA. In particular embodiments, the multianalyte sample includes protein biomarkers, cell-free DNA, and RNA (e.g., exosomal RNA). In particular embodiments, the multianalyte sample includes protein biomarkers, cell-free DNA, RNA (e.g., exosomal RNA), and lipids or metabolites. In particular embodiments, the multianalyte sample includes protein biomarkers, cell-free DNA, RNA (e.g., exosomal RNA), and lipids, and metabolites.

In various embodiments, sample 115A and/or sample 115B can be processed. For example, in various embodiments, sample 115A and/or sample 115B may be processed to extract specific analytes. In various embodiments, samples can undergo cellular disruption methods involving chemical methods or mechanical methods. Example chemical methods include osmotic shock, enzymatic digestion, detergents, or alkali treatment. Example mechanical methods include homogenization, ultrasonication or cavitation, pressure cell, or ball mill. In various embodiments, samples can undergo removal of membrane lipids or proteins or nucleic acid purification. Example chemical methods for removing membrane lipids or proteins and methods for nucleic acid purification include guanidine thiocyanate (GuSCN)-phenol-chloroform extraction, alkaline extraction, cesium chloride gradient centrifugation with ethidium bromide, Chelex® extraction, or cetyltrimethylammonium bromide extraction. Example physical methods for removing membrane lipids or proteins and methods for nucleic acid purification include solid-phase extraction methods using any of silica matrices, glass particles, diatomaceous earth, magnetic beads, anion exchange material, or cellulose matrix. Further details of nucleic acid extraction methods are described in Ali et al, Current Nucleic Acid Extraction Methods and Their Implications to Point-of-Care Diagnostics, Biomed Res. Int. 2017; 2017:9306564, which is hereby incorporated by reference in its entirety.

In various embodiments, as further shown in FIGS. 1A and 1B, sample 115A and/or sample 115B can be processed using an assay e.g., assay 120A or assay 120B. Assay 120A and assay 120B may, in various embodiments, be the same assay. In various embodiments, the assay 120A and assay 120B are different assays. Generally, an assay 120 is performed to generate data from a the sample 115. For example, an assay 120 may be an immunoassay for detecting expression levels of protein biomarkers. As another example, an assay 120 may include nucleic acid sequencing to generate sequence information of nucleic acids (e.g., cfDNA or RNA, such as exosomal RNA). As another example, an assay 120 may include performing mass spectrometry for detecting levels of metabolites and/or levels of lipids. Further details of example assays are described herein. As another example, an assay 120 can involve a microscope that captures images e.g., of a sample. For example, the microscope can be used to capture images of a tissue or of cells from a sample.

Generally, a screen is performed on marker information generated by the assay (e.g., assay 120A). In various embodiments, the screen is performed to determine whether a biological sample is at risk or not at risk of containing a signal indicative of cancer. For example, the screen is performed to determine whether a biological sample is at risk or not at risk of containing circulating tumor DNA. Circulating DNA within the biological sample may indicate that the individual (e.g., individual from whom the biological sample is obtained) may be at risk of cancer. In some scenarios, the screen 125 may determine that the subject 110 is negative for cancer (e.g., absence of cancer or lack of risk for cancer) and therefore, the subject 110 is reported as not at risk. In some scenarios, the subject 110 is reported as at risk for cancer (e.g., presence of cancer or presence of risk for cancer). As shown in FIG. 1A, the subject 110 can undergo a risk categorization 150 and can be assigned a frequency of healthcare utilization according to the risk categorization 150. As shown in FIG. 1B, the subject 110 can further undergo subsequent testing e.g., a Tier 2 test. For example, as shown in FIG. 1B, the subject 110 can undergo a second analysis 130 to determine if the subject 110 is positive or negative for cancer (e.g., presence or absence of cancer). In various embodiments, if the subject is positive for cancer, then the subject 110 is reported as having a presence of cancer. The subject can therefore receive care to treat and/or slow the progression of cancer. For example, the subject 110 can undergo, surgical resection, and/or therapeutic treatment, In various embodiments, if the subject is negative for cancer, then the subject 110 undergoes risk categorization 150. For example, the subject can be categorized into one of high risk, intermediate risk, or low risk, which corresponds to a frequency of healthcare utilization. Thus, these subjects 110 that are determined to have an absence of cancer need not all receive frequent future checkups and only those deemed to be lacking cancer, but at highest risk for developing cancer, can undergo more frequent future checkups.

In various embodiments, performing the screen 125 involves comparing the quantified values of analytes (e.g., any of nucleic acids, protein biomarkers, lipids, or metabolites) to one or more reference values or to threshold values. For example, a reference value can be a statistical measure of quantified values of analytes corresponding to individuals known to be at risk for cancer. Therefore, if the comparison identifies that the analytes for an individual is statistically significantly different from the reference value corresponding to individuals known to be at risk for cancer, then the screen can identify the subject 110 as not at risk for cancer.

In various embodiments, performing the assay 120A generates sequencing information for one or more genomic locations, such as one or more CpG islands. In various embodiments, performing the screen 125 involves comparing methylation information at one or more pre-selected genomic locations to quantified values of reference genomic locations. Based on the comparison, the screen 125 can identify the subject as at risk for cancer, or not at risk for cancer.

In various embodiments, the screen achieves at least 60% sensitivity in detecting presence of cancer. In various embodiments, the screen achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, the screen achieves at least 75% sensitivity. In particular embodiments, the screen achieves at least 76% sensitivity. In particular embodiments, the screen achieves at least 77% sensitivity. In particular embodiments, the screen achieves at least 78% sensitivity. In particular embodiments, the screen achieves at least 79% sensitivity. In particular embodiments, the screen achieves at least 80% sensitivity.

In various embodiments, the screen achieves at least 60% specificity in excluding individuals without cancer. In various embodiments, the screen achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, the screen achieves at least 90% specificity. In particular embodiments, the screen achieves at least 91% specificity. In particular embodiments, the screen achieves at least 92% specificity. In particular embodiments, the screen achieves at least 93% specificity. In particular embodiments, the screen achieves at least 94% specificity. In particular embodiments, the screen achieves at least 95% specificity.

In various embodiments, the screen achieves at least 15% positive predictive value. In various embodiments, the screen achieves at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% positive predictive value. In particular embodiments, the screen achieves at least 20% positive predictive value. In particular embodiments, the screen achieves at least 21% positive predictive value. In particular embodiments, the screen achieves at least 22% positive predictive value. In particular embodiments, the screen achieves at least 23% positive predictive value. In particular embodiments, the screen achieves at least 24% positive predictive value. In particular embodiments, the screen achieves at least 25% positive predictive value. In particular embodiments, the screen achieves at least 26% positive predictive value. In particular embodiments, the screen achieves at least 27% positive predictive value. In particular embodiments, the screen achieves at least 28% positive predictive value. In particular embodiments, the screen achieves at least 29% positive predictive value. In particular embodiments, the screen achieves at least 30% positive predictive value. In particular embodiments, the screen achieves at least 31% positive predictive value. In particular embodiments, the screen achieves at least 32% positive predictive value. In particular embodiments, the screen achieves at least 33% positive predictive value. In particular embodiments, the screen achieves at least 34% positive predictive value. In particular embodiments, the screen achieves at least 35% positive predictive value. In particular embodiments, the screen achieves at least 36% positive predictive value. In particular embodiments, the screen achieves at least 37% positive predictive value. In particular embodiments, the screen achieves at least 38% positive predictive value. In particular embodiments, the screen achieves at least 39% positive predictive value. In particular embodiments, the screen achieves at least 40% positive predictive value.

In various embodiments, the screen achieves at least 60% negative predictive value. In various embodiments, the screen achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In particular embodiments, the screen achieves at least 95% negative predictive value. In particular embodiments, the screen achieves at least 96% negative predictive value. In particular embodiments, the screen achieves at least 97% negative predictive value. In particular embodiments, the screen achieves at least 98% negative predictive value. In particular embodiments, the screen achieves at least 99% negative predictive value.

In various embodiments, performing a screen 125 comprises performing a family history evaluation, a life style evaluation, an age group, a demographic group, and/or a cancer test. In various embodiments, the cancer test is one or more of: a fecal immunochemical test of the initial stool sample, a fecal occult blood test, detection of blood and abnormal DNA in the initial stool sample, detection of ctDNA mutations and/or ctDNA fragment size and/or expression of proteins associated with cancer in the initial blood sample. In particular embodiments, the cancer test is a gastrointestinal (GI) cancer test that detects risk of a presence or absence of GI cancer. In various embodiments, the cancer test is a commercially available cancer test. Examples of a cancer diagnostic test include any of COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, imaware Prostate Cancer screening test, a PSA blood test, a mammogram, a colonoscopy, a Pap smear, am HPV test, a biopsy, a hereditary cancer screen (for example the Natera Empower test, Guardant Hereditary cancer test, Myriad Genetics hereditary cancer test or Labcorp hereditary cancer test), low-dose computed tomography (LDCT), a prenuvo cancer scan, or the Grail Galleri multi-cancer screening.

As shown in FIG. 1B, a sample 115B may be obtained from the subject 110 at a subsequent time if the screen 125 determines that the subject 110 is positive for a cancer. In various embodiments, the sample 115B may be obtained from the subject 110 e.g., by a medical professional during a visit at a reference laboratory or at a doctor's office. In various embodiments, the sample 115B may be obtained from the subject 110 during a visit at a doctor's office.

In various embodiments, after obtaining the sample 115B, methods involve performing one or more assays (e.g., assay 120B) on the sample 115B e.g., to generate data of one or more analytes (e.g., any of nucleic acids, protein biomarkers, lipids, or metabolites) in the sample 115B. Further details of exemplary assays are described herein.

Step 130 involves performing a second analysis to analyze the data of one or more analytes generated by the assay 120B. For example, performing the second analysis 130 may involve analyzing at least methylation statuses of nucleic acids of the sample 115B to determine whether the sample 115B has a presence or absence of a cancer signal. In various embodiments, performing the second analysis 130 may involve analyzing one or more additional analytes (e.g., any of nucleic acids, protein biomarkers, lipids, or metabolites) in the sample 115B.

In various embodiments, step 130 involves deploying a trained machine learning model to analyze data of one or more analytes. Methods for training and deploying a machine learning model are further described herein.

In various embodiments, following the screen (e.g., as shown in FIG. 1A) or after the second analysis 130 (e.g., as shown in FIG. 1B), the subject 110 can be deemed eligible for further follow up procedure. A further follow up procedure can include a future visit, the use of a specialist, a diagnostic testing, a diagnostic imaging, a biopsy, a medical appointment, and/or a prophylactic medication. In various embodiments, the frequency of the follow up procedure is determined for the subject 110 based on a risk categorization 150. Generally, the frequency of healthcare utilization for a higher risk subject (e.g., a subject at higher risk for cancer) is greater than the frequency of healthcare utilization for a lower risk subject (e.g., a subject at lower risk for cancer). For example, a higher risk subject may be recommended to obtain a colonoscopy as soon as possible, whereas a medium risk subject may be recommended to obtain a colonoscopy in 3-5 years and a low risk subject may be recommended to obtain a colonoscopy in 10 years.

As shown in FIG. 1A and FIG. 1B, methods involve performing a risk categorization 150 for the subject 110. In various embodiments, the risk categorization 150 is based on a score generated by the preceding step (e.g., the screen 125 in FIG. 1A, or the second analysis 130 in FIG. 1B). In various embodiments, the score is generated by deploying a trained machine learning model to analyze data of one or more analytes to determines a level of risk for the subject 110. In various embodiments, the level of risk is a score. In various embodiments, the score can be a single numerical value out of a fixed possible number of values. In various embodiments, the score can be a continuous value. For example, the score can be a value between 0 and 1, indicating a probabilistic value for the level of risk.

Generally, such a score can be correlated to a level of risk and therefore, is useful for assigning a risk categorization 150.

In various embodiments, the risk categorization 150 involves assigning the subject 110 to one of N possible categories. In various embodiments, N is any of 2, 3, 4, 5, 6, 7, 8, 9, 10, 11, 12, 13, 14, 15, 16, 17, 18, 19, 20, 21. 22. 23. 24, or 25. In particular embodiments, N is 3 and the risk categories can be any of low risk, intermediate risk, or high risk. In various embodiments, assigning the subject 110 to one of N possible categories involves comparing the score generated for the subject 110 (e.g., a score is generated by deploying a trained machine learning model to analyze data of one or more analytes) to one or more threshold scores. For example, threshold scores can differentiate between each of the N possible categories and therefore, if the score generated for the subject 110 falls within a certain predefined range that is defined by the threshold scores, then the subject 110 is assigned into a corresponding risk categorization 150.

### Methods for Analyzing Multianalytes for Prioritizing Healthcare Utilization

Disclosed herein are methods for analyzing data of one or more analytes from a sample obtained from a subject. In various embodiments, analyzing data of one or more analytes comprises implementing a machine learning model. In various embodiments, the machine learning model can be implemented when performing the screen e.g., screen 125 in FIG. 1A. In various embodiments, the machine learning model can be implemented when performing the second analysis e.g., second analysis 130 in FIG. 1B. The machine learning model may be trained to receive, as input, features of the multiple analytes. The machine learning model may generate a prediction that is informative for risk categorization.

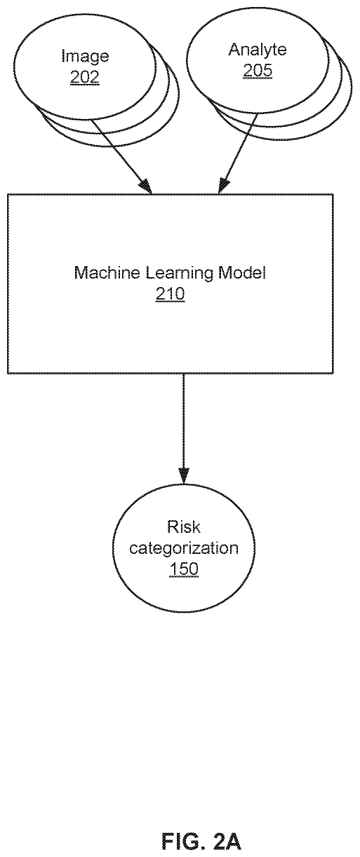

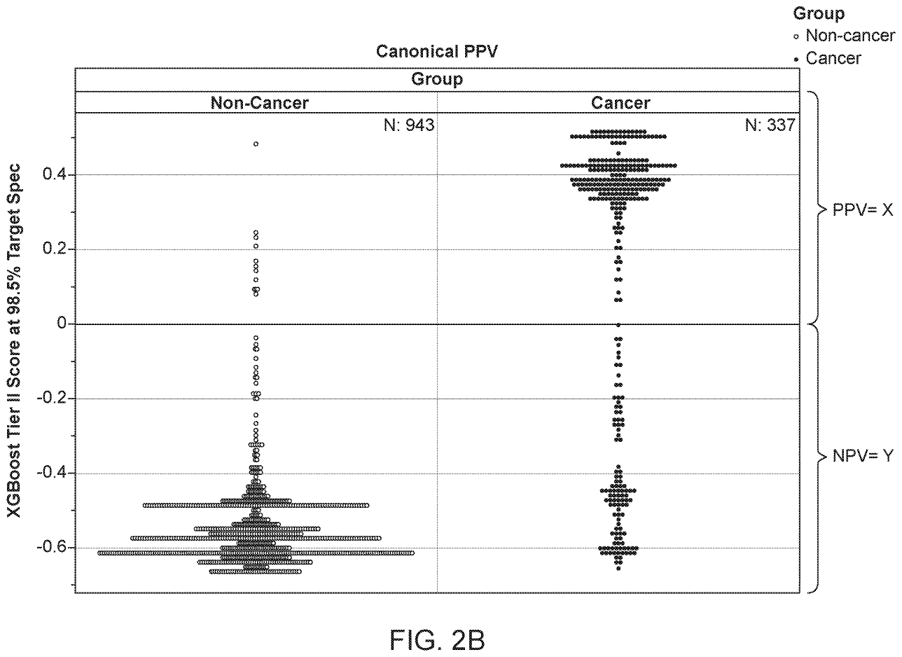

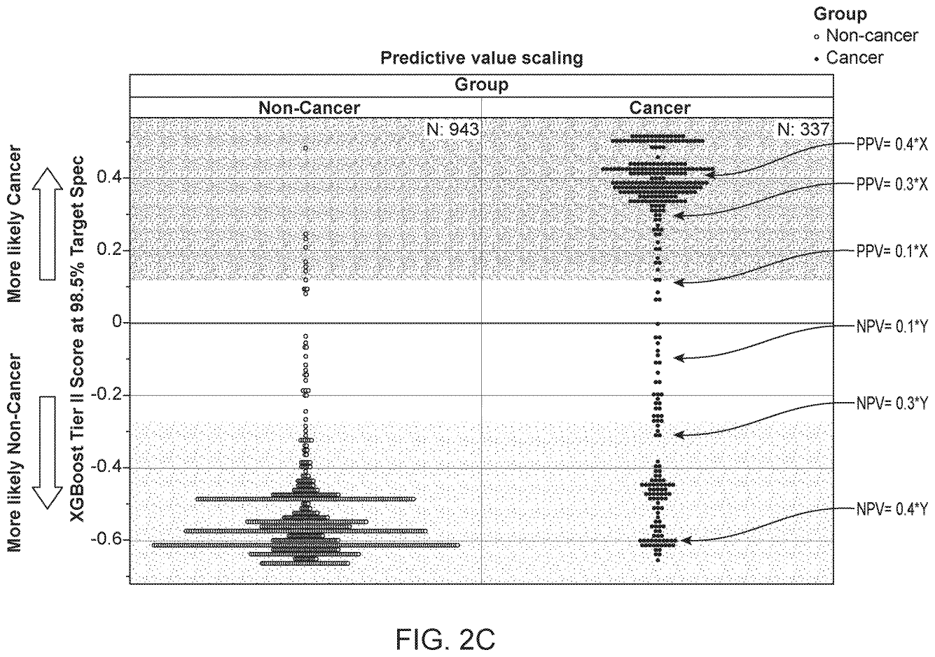

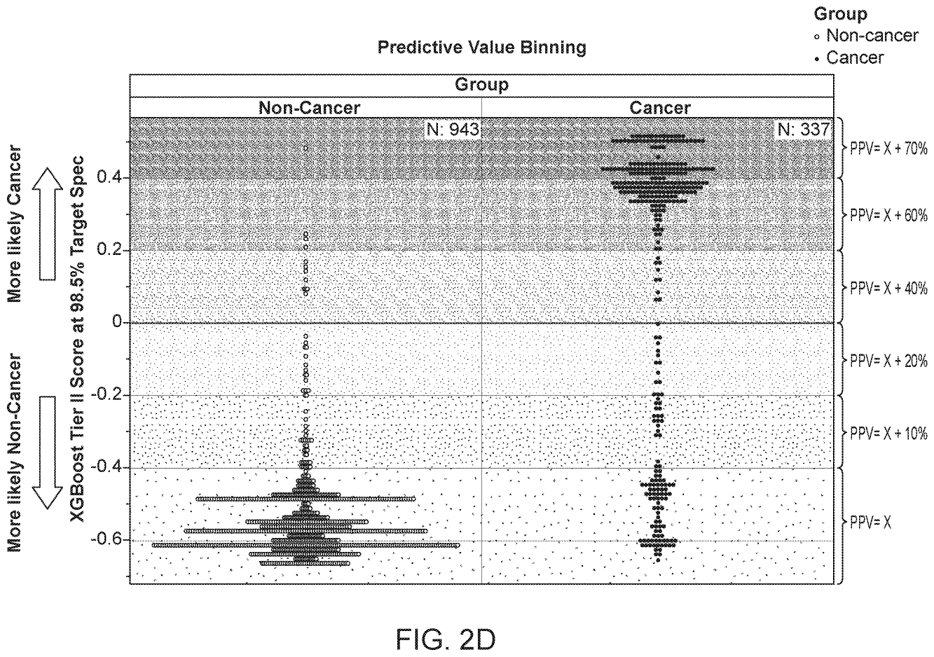

Reference is made to FIG. 2A, which shows an example flow diagram for analyzing multianalyte information, in accordance with an embodiment. FIG. 2A introduces a machine learning model 210, which receives as input features of analyte 205. In various embodiments, there may be additional analytes e.g., analyte 205A, analyte 205B, and analyte 205C (not explicitly shown in FIG. 2A). In various embodiments the machine learning model 210 may be trained to analyze fewer (e.g., one or two different analytes) or more (e.g., four, five, six, seven, eight, nine, ten, eleven, twelve, thirteen, fourteen, fifteen, sixteen, seventeen, eighteen, nineteen, or twenty different analytes). In particular embodiments, at least a first analyte is a protein analyte and a second analyte is a nucleic acid analyte (e.g., DNA or RNA). In various embodiments, the multianalyte information includes one or more protein analytes, one or more nucleic acid analytes (e.g., DNA or RNA), one or more lipids, and/or one or more metabolites.

In various embodiments, as shown in FIG. 2A, the machine learning model 210 may analyze one or more images 202. Here, the machine learning model 210 may analyze one or more images 202 to generate the prediction of the risk categorization 150. Further details of example images 202 (e.g., microscopy images) are described herein. In various embodiments, the machine learning model 210 analyzes one or more images 202 and one or more analytes 205. Thus, the combination of the one or more images 202 and one or more analytes 205 enables the machine learning model 210 to better predict a risk categorization 150.

In particular embodiments, a nucleic acid analyte is cfDNA. In such embodiments, the machine learning model may receive input features of sequencing information of the cfDNA. For example, the sequencing information of the cfDNA may, in various embodiments, include methylation information of the cfDNA. The methylation information of the cfDNA may be informative for determining risk of cancer.

As an example, the methylation information of cfDNA may include methylation information for one or more pre-selected genomic locations. In various embodiments, the pre-selected genomic locations may refer to cancer informative CGIs, which can be values that are provided as input to the machine learning model. The machine learning model provides a predicted output that is informative for performing risk categorization based on the values of the cancer informative CGIs. Example CGIs are disclosed in WO2018209361 (see Table 1), WO2022133315 (see Table 2 entitled “TOO Methylation Sites” and Table 3 entitled “Pan Cancer Methylation Sites”), and WO2023147567 (see Tables 1-4), each of which is hereby incorporated by reference in its entirety. Further example CGIs are disclosed in Tables 4-9 included herein. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed.

In various embodiments, the analysis involves analyzing at least 100 CGIs. In various embodiments, the analysis involves analyzing at least 100 CGIs, at least 150 CGIs, at least 200 CGIs, at least 300 CGIs, at least 400 CGIs, at least 500 CGIs, at least 600 CGIs, at least 700 CGIs, at least 800 CGIs, at least 900 CGIs, at least 1000 CGIs, at least 1500 CGIs, at least 2000 CGIs, at least 2500 CGIs, at least 3000 CGIs, at least 3500 CGIs, at least 4000 CGIs, at least 4500 CGIs, at least 5000 CGIs, at least 5500 CGIs, or at least 6000 CGIs. In particular embodiments, performing the screen involves analyzing at least 500 CGIs. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed.

In various embodiments, the machine learning model analyzes protein biomarker data. In various embodiments, the machine learning model analyzes M protein biomarkers. In various embodiments, the M protein biomarkers include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 protein biomarkers.

In various embodiments, the machine learning model analyzes exosomal RNA markers. In various embodiments, the machine learning model analyzes marker data of cancer specific biomarkers. In various embodiments, the machine learning model analyzes marker data of non-cancer specific biomarkers. In various embodiments, the machine learning model analyzes marker data of constitutive cancer specific markers. In various embodiments, the prediction model analyzes marker data of cancer specific biomarkers and non-cancer specific biomarkers. In various embodiments, the prediction model analyzes marker data of cancer specific biomarkers and constitutive cancer specific markers. In various embodiments, the prediction model analyzes marker data of non-cancer specific biomarkers and constitutive cancer specific markers. In various embodiments, the prediction model analyzes marker data of cancer specific markers, non-cancer specific biomarkers, and constitutive cancer specific markers. Cancer specific markers are disclosed in Table 1. Non-cancer specific markers are disclosed in Table 2. Constitutive cancer specific markers are disclosed in Table 3.

In various embodiments, the machine learning model analyzes Nexosomal RNA (exoRNA) markers. In various embodiments, the NexoRNA markers include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 exoRNA markers (e.g., exoRNA markers disclosed in any of Tables 1, 2, 3).

In various embodiments, the machine learning model analyzes values of one or more metabolites and/or one or more lipids. In various embodiments, the machine learning model analyzes X different metabolites and/or lipids. In various embodiments, the X metabolites and/or lipids include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 metabolites and/or lipids.

In various embodiments, the machine learning model achieves at least 60% sensitivity in detecting presence of a cancer signal. In various embodiments, the analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, the analysis achieves at least 85% sensitivity. In particular embodiments, the analysis achieves at least 86% sensitivity. In particular embodiments, the analysis achieves at least 87% sensitivity. In particular embodiments, the analysis achieves at least 88% sensitivity. In particular embodiments, the analysis achieves at least 89% sensitivity. In particular embodiments, the analysis achieves at least 90% sensitivity.

In various embodiments, the analysis achieves at least 60% specificity in excluding individuals without a cancer signal. In various embodiments, the analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, the analysis achieves at least 90% specificity. In particular embodiments, the analysis achieves at least 91% specificity. In particular embodiments, the analysis achieves at least 92% specificity. In particular embodiments, the analysis achieves at least 93% specificity. In particular embodiments, the analysis achieves at least 94% specificity. In particular embodiments, the analysis achieves at least 95% specificity.

In various embodiments, the analysis achieves at least 50% positive predictive value. In various embodiments, the analysis achieves at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value. In particular embodiments, the analysis achieves at least 80% positive predictive value. In particular embodiments, the analysis achieves at least 81% positive predictive value. In particular embodiments, the analysis achieves at least 82% positive predictive value. In particular embodiments, the analysis achieves at least 83% positive predictive value. In particular embodiments, the analysis achieves at least 84% positive predictive value. In particular embodiments, the analysis achieves at least 85% positive predictive value.

In various embodiments, the analysis achieves at least 60% negative predictive value. In various embodiments, the analysis achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In particular embodiments, the analysis achieves at least 90% negative predictive value. In particular embodiments, the analysis achieves at least 91% negative predictive value. In particular embodiments, the analysis achieves at least 92% negative predictive value. In particular embodiments, the analysis achieves at least 93% negative predictive value. In particular embodiments, the analysis achieves at least 94% negative predictive value. In particular embodiments, the analysis achieves at least 95% negative predictive value. In particular embodiments, the analysis achieves at least 96% negative predictive value. In particular embodiments, the analysis achieves at least 97% negative predictive value. In particular embodiments, the analysis achieves at least 98% negative predictive value. In particular embodiments, the analysis achieves at least 99% negative predictive value.

### Patient Stratification for Prioritizing Healthcare Utilization

In various embodiments, the subject 110 is assigned into a corresponding risk categorization 150 based on scores corresponding to certain positive predictive value (PPV). For example, assigned scores of subjects can be compared against a threshold value for categorizing the subjects into a category. The precision of that categorization can be expressed as a PPV. Thus, the assigned scores of subjects can correspond to a particular PPV. FIG. 2B shows canonical positive predictive value (PPV) thresholding, in accordance with an embodiment. For example, given a threshold score of 0, subjects assigned a score greater than 0 are categorized as “cancer” whereas subjects assigned a score less than 0 are categorized as “non-cancer.” The PPV can be a measure of precision of the subjects categorized in the “cancer” category. Similarly, the negative predictive value (NPV) can be a measure of the subjects categorized in the “non-cancer” category.

In various embodiments, the subject 110 is assigned into a corresponding risk categorization 150 based on a scaling value, such as a predictive scaling value measured by a scaled PPV. Subjects with scores that correspond to higher scaled PPVs can be assigned to a higher risk category whereas subjects with scores that correspond to lower scaled PPVs can be assigned to a lower risk category. FIG. 2C shows an example patient stratification based on predictive value scaling, in accordance with an embodiment. Here, subjects are assigned scores that correspond to a continuous scale of PPVs. For example, certain subjects that are more likely cancerous may be assigned scores that correspond to higher PPV (e.g., PPV=0.4*X or PPV=0.3*X). Certain subjects that are likely cancerous may be assigned scores that correspond to lower PPV (but are still likely to be cancerous) (e.g., PPV=0.2*X or PPV=0.1*X). In this example, subjects corresponding to the higher PPV (e.g., PPV=0.4*X or PPV=0.3*X) may be assigned to a higher risk category than subjects corresponding to the lower PPV (e.g., PPV=0.2*X or PPV=0.1*X). Similarly, subjects that are less likely cancerous may be assigned scores that correspond to higher NPV (e.g., NPV=0.4*Y or NPV=0.3*Y). Certain subjects that are likely non-cancerous may be assigned scores that correspond to lower NPV (but are still likely to be non-cancerous) (e.g., NPV=0.2*X or NPV=0.1*X). In this example, subjects corresponding to the higher NPV (e.g., NPV=0.4*X or NPV=0.3*X) may be assigned to a lower risk category than subjects corresponding to the lower NPV (e.g., NPV=0.2*X or NPV=0.1*X).

Altogether, the scaled PPV could be useful for assigning subject to risk categorizations for follow up steps. For example a subject found to be associated with a high scaled PPV may need more extensive follow up than a subject found to be associated with a weakly positive scaled PPV. Conversely a subject associated with a low NPV may require more frequent longitudinal surveillance than other subjects associated with a higher NPV. In various embodiments, scaled predictive values could be used to identify appropriate confirmatory testing. A subject associated with a high PPV may not need any confirmation but a patient associated with a weakly positive PPV could be assigned for orthogonal confirmation.

In various embodiments, the subject 110 is assigned into a corresponding risk categorization 150 based on a binned value, such as a predictive PPV bin. FIG. 2D shows an example patient stratification based on predictive value binning, in accordance with an embodiment. Here, the baseline PPV can be set at “X” and therefore, subjects assigned scores that correspond to this baseline PPV can be binned in a category. As shown in FIG. 2D, additional bins can be generated, with each additional bin including subjects assigned scores that correspond to higher PPVs. For example, a second bin includes subjects with assigned scores that correspond to PPV=X+10%. An additional bin includes subjects with assigned scores that correspond to PPV=X+20%. An additional bin includes subjects with assigned scores that correspond to PPV=X+40%. An additional bin includes subjects with assigned scores that correspond to PPV=X+60%. An additional bin includes subjects with assigned scores that correspond to PPV=X+70%. The particular binning values can differ than shown in FIG. 2D.

In various embodiments, the risk categorization 150 involves assigning the subject 110 to one of N possible categories based on a predicted cancer incidence. For example, the risk categorization 150 can involve assigning the subject 110 into a high risk category if the predicted cancer incidence is above a threshold cancer incidence level. As another example, the risk categorization 150 can involve assigning the subject 110 into a low risk category if the predicted cancer incidence is below a threshold cancer incidence level. In various embodiments, the threshold cancer incidence level is 75% incidence.

Generally, based on the risk categorization 150, the subject 110 is assigned a particular healthcare utilization 175 level. For example, a level of healthcare utilization 175 can be a frequency of healthcare utilization. In various embodiments, healthcare utilization comprises a medical procedure, a future visit to a hospital, the use of a specialist, a diagnostic testing, a diagnostic imaging, a biopsy, a medical appointment, and/or a prophylactic medication. In particular embodiments, healthcare utilization refers to a medical procedure, such as a colonoscopy. In particular embodiments, healthcare utilization refers to a fecal immunochemical test. In particular embodiments, healthcare utilization refers to a diagnostic imaging, examples of which include an ultrasound scan, a MRI scan, or a CT scan. Generally, a subject 110 assigned to a risk category of higher risk would be further assigned to a higher level of healthcare utilization in comparison to a subject assigned to a risk category of lower risk.

In various embodiments, if the subject 110 is assigned to a high risk category, then the subject 110 can be assigned a frequency of healthcare utilization of less than 1 year. For example, one or more follow up procedures for the subject 110 (a future visit, the use of a specialist, a diagnostic testing, a diagnostic imaging, a biopsy, a medical appointment, and/or a prophylactic medication) is performed at a frequency of 1 year or less. In various embodiments, one or more follow up procedures for the subject 110 (a future visit, the use of a specialist, a diagnostic testing, a diagnostic imaging, a biopsy, a medical appointment, and/or a prophylactic medication) is performed at a frequency of 11 months or less, 10 months or less, 9 months or less, 8 months or less, 7 months or less, 6 months or less, 5 months or less, 4 months or less, 3 months or less, 2 months or less, or 1 month or less.

In various embodiments, if the subject 110 is assigned to an intermediate risk category, then the subject 110 can be assigned a frequency of healthcare utilization of between 1 year and 2 years. For example, one or more follow up procedures for the subject 110 (a future visit, the use of a specialist, a diagnostic testing, a diagnostic imaging, a biopsy, a medical appointment, and/or a prophylactic medication) is performed at a frequency of between 1 and 2 years (e.g., frequency of 12 months, 13 months, 14 months, 15 months, 16 months, 17 months, 18 months, 19 months, 20 months, 21 months, 22 months, 23 months, or 24 months).

In various embodiments, if the subject 110 is assigned to an intermediate risk category, then the subject 110 can be assigned a frequency of healthcare utilization of greater than 2 years. For example, one or more follow up procedures for the subject 110 (a future visit, the use of a specialist, a diagnostic testing, a diagnostic imaging, a biopsy, a medical appointment, and/or a prophylactic medication) is performed at a frequency of between 2 years and 5 years. In various embodiments, one or more follow up procedures for the subject 110 (a future visit, the use of a specialist, a diagnostic testing, a diagnostic imaging, a biopsy, a medical appointment, and/or a prophylactic medication) is performed at a frequency of 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months.

For a subject 110 with a particular assigned frequency of healthcare utilization, the subject 110 can continue to undergo one or more follow up procedures at the identified frequency until a triggering event occurs. For example, a triggering event can be a diagnosis of cancer, a progression of cancer, an increasing severity of a cancer, or a changing stage of a cancer.

In particular embodiments, the patients who have not been identified as high risk are the important patients to track. Given that the large majority of patients are unlikely to be identified as high risk (e.g., given low incidence rate of cancer), then it is beneficial to triage this large population of patients to ensure that healthcare resources are efficiently used. For example, patients that are not identified as high risk can include patients that are categorized as intermediate risk or low risk.

In various embodiments, methods involve performing longitudinal tracking of patients, such as patients not identified as high risk, based on the assigned frequency of healthcare utilization. Thus, over time, as long as a patient is not categorized as high risk, the patient can continue to be followed up at a determined frequency to longitudinally track that patient. In various embodiments, the patient can be longitudinally tracked over at least 2 timepoints, at least 3 timepoints, at least 4 timepoints, at least 5 timepoints, at least 6 timepoints, at least 7 timepoints, at least 8 timepoints, at least 9 timepoints, at least 10 timepoints, at least 11 timepoints, at least 12 timepoints, at least 13 timepoints, at least 14 timepoints, at least 15 timepoints, at least 16 timepoints, at least 17 timepoints, at least 18 timepoints, at least 19 timepoints, or at least 20 timepoints. In various embodiments, the patient can be longitudinally tracked for between 1 and 50 time points. In various embodiments, the patient can be longitudinally tracked for between 2 and 20 time points. In various embodiments, each timepoint can be separated from a prior timepoint based on the determined frequency of healthcare utilization assigned for the patient at the prior timepoint (e.g., by performing the methods disclosed herein). For example, at timepoint 1, methods may involve analyzing one or more analytes of the patient e.g., using a machine learning model, to determine a risk score. The patient is categorized based on the risk score as non-high risk and is provided a follow up procedure at a second timepoint. Thus, at the second time point, the methods can be repeated to analyze one or more analytes of the patient e.g., using a machine learning model, to determine an updated risk score. The patient is categorized based on the updated risk score as non-high risk and is provided a provided a follow up procedure at a third timepoint. Thus, the process can continue to repeat in this manner. At each timepoint, methods can involve determining a risk score for the patient and therefore, the determined risk score at each time point can serve as the basis for longitudinally tracking the patient. For example, in addition to tracking a patient's risk category over time, the above methods may be used to track response to therapeutics, e.g. longitudinally tracking whether a patient's risk score lowers with therapeutic intervention. In some scenarios, real-time monitoring of a patient can be performed where methods can involve repeating the analysis of one or more analytes at subsequent time points to continuously determine updated risk scores. Thus, the continuously determined updated risk scores can serve as the basis for understanding the changing risk for the patient.

In various embodiments, methods involve performing longitudinal tracking of patients who have a familial history of cancer. In various embodiments, patients who have a familial history of cancer can refer to individuals with at least one direct family relative, such as a first-degree relative (e.g., sibling, parent, children) who was previously diagnosed with cancer. In various embodiments, patients who have a familial history of cancer can refer to individuals with at least one direct family relative, such as a first-degree relative (e.g., sibling, parent, children) who was previously diagnosed with cancer prior to a threshold age. In various embodiments, the threshold age is 30 years old, 31 years old, 32 years old, 33 years old, 34 years old, 35 years old, 36 years old, 37 years old, 38 years old, 39 years old, 40 years old, 41 years old, 42 years old, 43 years old, 44 years old, 45 years old, 46 years old, 48 years old, 49 years old, 50 years old, 51 years old, 52 years old, 53 years old, 54 years old, 55 years old, 56 years old, 57 years old, 58 years old, 59 years old, 60 years old, 61 years old, 62 years old, 63 years old, 64 years old, 65 years old, 66 years old, 67 years old, 68 years old, 69 years old, or 70 years old. In particular embodiments, the threshold age is 45 years old. In particular embodiments, the threshold age is 50 years old. In particular embodiments, the threshold age is 55 years old.

### Machine Learning Model

Disclosed herein are machine learning models that may analyze input features of one or more analytes to generate a prediction, such as a risk score. Thus, the risk score can be used for categorizing a patient from whom the one or more analytes were obtained.

In various embodiments, a machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks).

The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

In various embodiments, a machine learning model is trained specifically for a particular stage of cancer. For example, the machine learning model can be trained using training data obtained from one or more patients who are known to have a particular stage of cancer. For example, a machine learning model can be trained using training data obtained from patients who are known to have Stage I cancer. For example, a machine learning model can be trained using training data obtained from patients who are known to have Stage II cancer. For example, a machine learning model can be trained using training data obtained from patients who are known to have Stage III cancer. For example, a machine learning model can be trained using training data obtained from patients who are known to have Stage IV cancer. In particular embodiments, multiple different machine learning models are trained such that each model is trained using training data obtained from patients who are know to have a particular stage of cancer. For example, at least four different machine learning models may be trained using training data obtained from patients known to have Stage I cancer, patients known to have Stage II cancer, patients known to have Stage III cancer, and patients known to have Stage IV cancer, respectively. Analytes may be differentially present depending on the stage of cancer, and therefore, it may be valuable to develop different models trained for particular stages of cancer. For example, the methylation from an adenoma may have sharper, more definitive methylation states at later stages of cancer in comparison to earlier stages of cancer.

### Flow Diagram for Prioritizing Healthcare Utilization

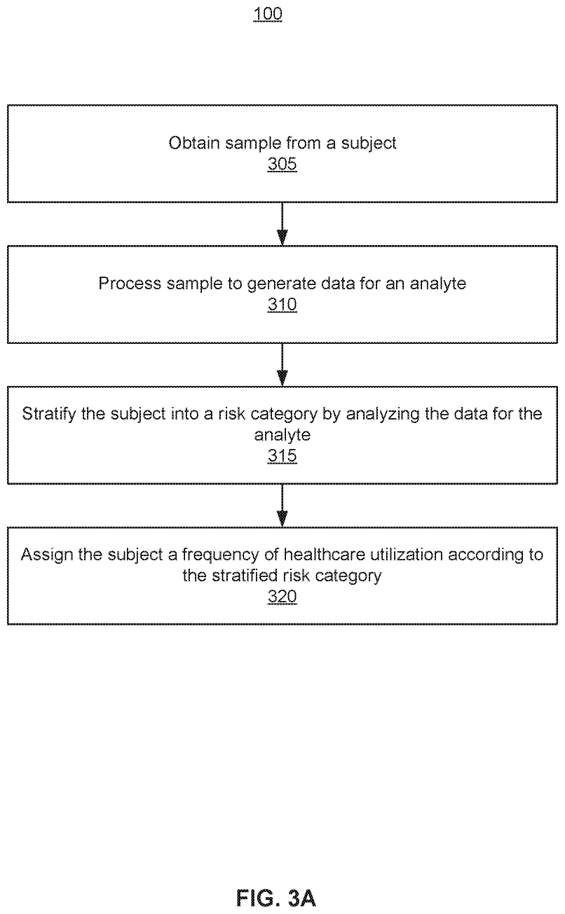

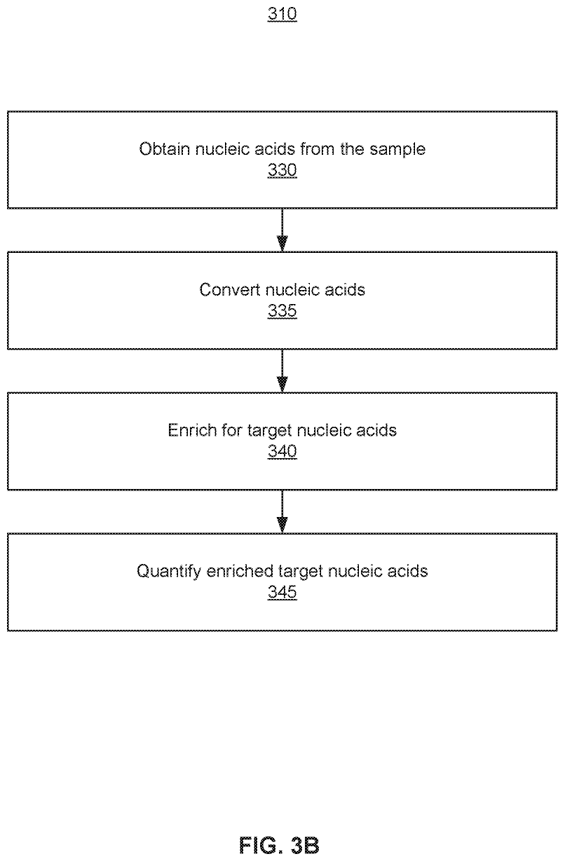

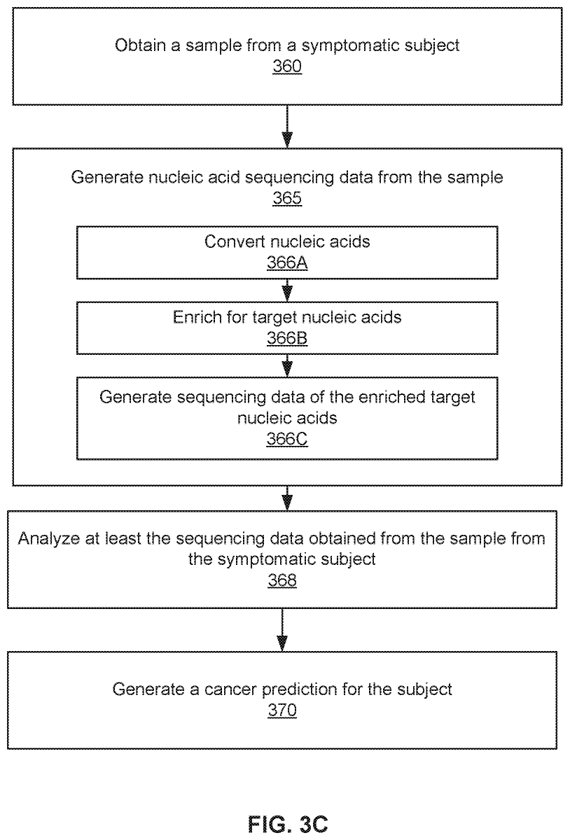

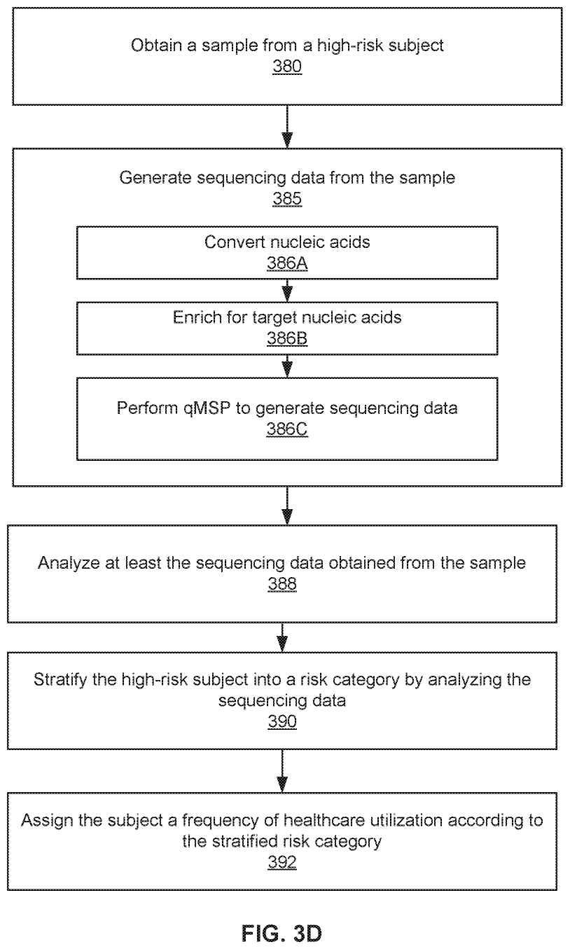

FIG. 3A shows an example flow process for prioritizing healthcare resources in cancer care, in accordance with an embodiment.

Step 305 involves obtaining a sample from a subject, such as a multianalyte sample.

Step 310 involves processing the sample to generate data of one or more analytes. In various embodiments, step 310 involves processing the sample to generate data for at least one or more protein analytes and one or more nucleic acid analytes. In various embodiments, the nucleic acid analytes comprise cfDNA. In various embodiments, the nucleic acid analytes comprise RNA. In various embodiments, the nucleic acid analytes comprise nucleic acids from exosomes. In various embodiments, step 310 further involves processing the multianalyte sample to generate data for one or more additional analytes, examples of which include lipids and metabolites.

Step 315 involves stratifying the subject into a risk category by analyzing the generated data (e.g., data for at least one or more protein analytes and one or more nucleic acid analytes). For example, step 315 can involve generating a risk score for the subject based on the analysis of the generated data.

Step 320 involves assigning the subject a frequency of healthcare utilization according to the stratified risk category. For example, the stratified risk category can be one of a high risk, intermediate risk, or low risk. The frequency of healthcare utilization determined for the subject in a high risk category is greater than the frequency of healthcare utilization determined for a subject in the intermediate risk category and/or the low risk category.

Reference is further made to FIG. 3B, which shows an example flow process for processing a sample to generate data for an analyte, in accordance with step 310 of FIG. 3A.

Step 330 involves obtaining nucleic acids from the sample. For example, nucleic acids, such as cfDNA, can be extracted from the sample using methods disclosed herein.

Step 335 involves converting the nucleic acids. Example methods for converting the nucleic acids include performing any of bisulfite conversion, enzymatic conversion, or nitrite conversion. In particular embodiments, step 335 involves performing bisulfite conversion to convert the extracted nucleic acids.

Step 340 involves enriching for target nucleic acids. For example, step 340 can involve performing nucleic acid amplification or hybrid capture to enrich for target nucleic acids. A target nucleic acid can be a converted sequence derived from a fully methylated, a partially methylated, or a fully unmethylated sequence. In particular embodiments, step 340 involves providing one or more nucleic acid probes designed to hybridize to a target sequence (or one or more sequences upstream or downstream to a target sequence) of target nucleic acids. Thus, the nucleic acid probes can be used to enrich for the target nucleic acids. For example, nucleic acid probes can be used to initiate nucleic acid amplification to amplify the target nucleic acids. As another example, nucleic acid probes can be biotinylated and after hybridizing to a target sequence or one or more sequences upstream or downstream to a target sequence) of target nucleic acids, the nucleic acid probes and be bound by streptavidin modified beads to enrich for the target nucleic acids.

Step 345 involves quantifying the enriched target nucleic acids. In particular embodiments, step 345 involves performing a quantitative assay that is quick and cost-effective. In various embodiments, such a quantitative assay can include quantitative PCR (qPCR) or digital PCR (dPCR). In particular embodiments, the quantitative assay involves a qMSP assay. In particular embodiments, step 345 does not involve performing sequencing.

### Additional Example Flow Diagrams

FIG. 3C shows an example flow process for generating a cancer prediction for a subject, in accordance with an embodiment.

Step 360 involves obtaining a sample from a symptomatic subject. In various embodiments, a symptomatic subject may be an individual exhibiting symptoms of a cancer. In various embodiments, a symptomatic subject may be exhibiting symptoms of a cancer, but has not yet been diagnosed with cancer. In various embodiments, a symptomatic subject may have undergone a previous test or screen (such as a screen 125 shown in FIG. 1A or FIG. 1B) and was identified as exhibiting symptoms.

Step 365 involves generating nucleic acid sequencing data from the sample. As shown in FIG. 3C, step 365 includes substeps of steps 366A, 366B, and 366C.

Step 366A involves converting the nucleic acids. Example methods for converting the nucleic acids include performing any of bisulfite conversion, enzymatic conversion, or nitrite conversion. In particular embodiments, step 366A involves performing bisulfite conversion to convert the extracted nucleic acids.

Step 366B involves enriching for target nucleic acids. For example, step 366B can involve performing nucleic acid amplification or hybrid capture to enrich for target nucleic acids. A target nucleic acid can be a converted sequence derived from a fully methylated, a partially methylated, or a fully unmethylated sequence.

Step 366C involves generating sequencing data of the enriched target nucleic acids. In particular embodiments, step 366C involves performing a sequencing assay to generate the sequencing data. For example, step 366C can involve performing next generation sequencing (NGS). In some embodiments, step 366C can include performing quantitative PCR (qPCR) or digital PCR (dPCR). In particular embodiments, step 366C involves performing a qMSP assay.

Step 368 involves analyzing at least the sequencing data obtained from the sample from the symptomatic subject. In various embodiments, step 368 involves analyzing data from one or more additional analytes (e.g., any of proteins, metabolites, lipids, and/or nucleic acids (e.g., DNA such as cell-free DNA or RNA)). In various embodiments, step 368 involves implementing a machine learning model to analyze the sequencing data and/or data from one or more additional analytes.

Step 370 involves generating a cancer prediction for the subject. In various embodiments, the cancer prediction is a presence or absence of a cancer. In various embodiments, the cancer prediction is a presence or absence of a GI cancer (e.g., colorectal, gastroesophageal (GEJ), esophageal, stomach, liver/biliary, pancreas, or gallbladder). In various embodiments, the cancer prediction is a presence or absence of one or more of a colorectal cancer, esophageal cancer, GEJ cancer, stomach cancer, liver cancer, biliary cancer, pancreatic cancer, gall bladder cancer, lung cancer, head and neck cancer, ovarian cancer, or lymphoma.

Reference is now made to FIG. 3D shows an example flow process for prioritizing healthcare resources by analyzing qMSP sequencing data of a high-risk subject, in accordance with an embodiment.

Step 380 involves obtaining a sample from a high-risk subject. In various embodiments, the high-risk subject underwent a prior test that revealed a positive cancer result. A prior test can include any of COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, imaware Prostate Cancer screening test, a PSA blood test, a mammogram, a colonoscopy, a Pap smear, am HPV test, a biopsy, a hereditary cancer screen (for example the Natera Empower test, Guardant Hereditary cancer test, Myriad Genetics hereditary cancer test or Labcorp hereditary cancer test), low-dose computed tomography (LDCT), a prenuvo cancer scan, or the Grail Galleri multi-cancer screening.

Step 385 involves generating sequencing data from the sample. As shown in FIG. 3D, step 385 includes substeps of steps 386A, 386B, and 386C.

Step 386A involves converting the nucleic acids. Example methods for converting the nucleic acids include performing any of bisulfite conversion, enzymatic conversion, or nitrite conversion. In particular embodiments, step 386A involves performing bisulfite conversion to convert the extracted nucleic acids.

Step 386B involves enriching for target nucleic acids. For example, step 386B can involve performing nucleic acid amplification or hybrid capture to enrich for target nucleic acids. A target nucleic acid can be a converted sequence derived from a fully methylated, a partially methylated, or a fully unmethylated sequence.

Step 386C involves performing quantitative methylation-specific PCR (qMSP) assay to generate sequencing data. Generally, performing a qMSP can be quicker and more cost-effective in comparison to performing sequencing. Thus, a qMSP assay can be a preferred assay to quickly generate sequencing data.

Step 388 involves analyzing at least the sequencing data obtained from the sample. In various embodiments, step 388 involves analyzing data from one or more additional analytes (e.g., any of proteins, metabolites, lipids, and/or nucleic acids (e.g., DNA such as cell-free DNA or RNA)). In various embodiments, step 388 involves implementing a machine learning model to analyze the sequencing data and/or data from one or more additional analytes.

Step 390 involves stratifying the subject into a risk category by analyzing the generated data (e.g., data for at least one or more protein analytes and one or more nucleic acid analytes). For example, step 390 can involve generating a risk score for the subject based on the analysis of the generated data.

Step 392 involves assigning the subject a frequency of healthcare utilization according to the stratified risk category. For example, the stratified risk category can be one of a high risk, intermediate risk, or low risk. The frequency of healthcare utilization determined for the subject in a high risk category is greater than the frequency of healthcare utilization determined for a subject in the intermediate risk category and/or the low risk category.

### Cancers

Methods disclosed herein are useful for prioritizing healthcare resources in cancer care. Specifically, methods involve analyzing multi-analyte samples obtained from subjects and categorizing subjects into risk categories. Thus, subjects in higher risk categories undergo more frequent tracking and/or follow up whereas subjects in lower risk categories undergo less frequent tracking and/or follow up, thereby enabling prioritization of healthcare resources for those subjects that are most at risk of cancer.

In various embodiments, methods disclosed herein are useful for prioritizing healthcare resources in scenarios where the cancer is an early stage cancer. In various embodiments, the cancer is a preclinical phase cancer. In various embodiments, the cancer is a stage I cancer. In various embodiments, the cancer is a stage II cancer.

In various embodiments, the cancer is any of an acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

In various embodiments, methods disclosed herein are useful for prioritizing healthcare resources when providing care for patients with gastrointestinal (GI) cancer. Examples of GI cancer include esophageal cancer, gastric cancer, colorectal cancer, and anal cancer. In particular embodiments, methods disclosed herein are useful for prioritizing healthcare resources when providing care for patients with colorectal cancer.

In particular embodiments, the methods disclosed herein are useful for prioritizing healthcare resources when providing care for patients with breast cancer. are useful for prioritizing healthcare resources when providing care for patients with lung cancer.

### Assays

Methods disclosed herein involve performing an assay e.g., assay 120A or assay 120B to generate data for one or more analytes. Assays described in this section can be deployed to analyze either of the sample 115A or the sample 115B shown in FIGS. 1A and 1B.

### Assays for Generating Imaging Data

In various embodiments, methods involve analyzing imaging data, such as microscopy data captured from a microscope. Thus, microscopy data captured from microscopy images can serve as features for a machine learning model. Such a machine learning model can analyze imaging data to categorize the subject into a risk category for purposes of assigning a frequency of healthcare utilization for the subject. In various embodiments, microscopy data can be captured using a variety of different imaging modalities including confocal microscopy, super-high-resolution microscopy, in vivo two photon microscopy, electron microscopy (e.g., scanning electron microscopy or transmission electron microscopy), atomic force microscopy, bright field microscopy, and phase contrast microscopy.

In some scenarios, microscopy data are captured of cells from a subject of interest. Such cells may be cultured (e.g., in vitro) or may have been obtained and fixed e.g., on a tissue slide. In some scenarios, microscopy data are captured of a tissue obtained from a subject of interest. For example, cells or a tissue can be obtained and processed e.g., stained using primary and/or secondary antibodies that are fluorescently tagged. Thus, details of the cells and/or tissue can be observed through the fluorescent signals of the tags. In various embodiments, confocal microscopy is employed and tissues or tissue organoids are embedded in optimal tissue cutting compound and frozen at −20° C. Once frozen, tissues are sliced using a microtome and tissue slices are mounted on glass slides. Tissue slices are stained and fixed to prepare them for imaging. Tissue slices can be imaged using fluorescent (e.g., confocal) microscopy.

For immunohistochemistry, tissues are fixed, paraffin embedded, and cut. Generally, tissues are fixed using a formaldehyde fixation solution. Tissues are dehydrated by immersing them consecutively in increasing concentrations of ethanol (e.g., 70%, 90%, 100% ethanol) and then immersed in xylene. Tissues are embedded in paraffin and then cut into tissue sections (e.g., 5-15 microns in thickness). This can be accomplished using a microtome. Tissue sections are mounted onto histological slides, and then dried. Paraffin embedded sections can then be stained for particular targets (e.g., proteins, biomarkers) of interest. Sections are rehydrated (e.g., in decreasing concentrations of ethanol—100%, 95%, 70%, and 50% ethanol) and then rinsed with deionized H₂O. If needed, tissues are treated using blocking buffer to block for non-specific staining between a primary antibody and the tissue. Example blocking buffer can include 1% horse serum in phosphate buffered saline. Primary antibodies are diluted to appropriate dilutions and applied to the tissue sections. Tissue slices are washed, then incubated with a secondary antibody specific for the primary antibody. Tissue slices are washed, and then mounted. Tissue slices can then be imaged using microscopy (e.g., bright field microscopy, phase contrast microscopy, or fluorescence microscopy). Additional methods for performing immunohistochemistry are described in further detail in Simon et al, BioTechniques, 36 (1): 98 (2004) and Haedicke et al., BioTechniques, 35 (1): 164 (2003), each of which is hereby incorporated by reference in its entirety. In various embodiments, immunohistochemistry can be automated using commercially available instruments, such as the Benchmark ULTRA system available from the Roche Group.

### Assays for Generating Data for Protein Biomarkers

In various embodiments, performing an assay can involve performing an assay to generate protein biomarker data (e.g., protein expression levels). One approach for measuring protein expression levels is to perform protein identification with the use of antibodies. As used herein, the term “antibody” is intended to refer broadly to any immunologic binding agent such as IgG, IgM, IgA, IgD and IgE. Generally, IgG and/or IgM are the most common antibodies in the physiological situation and are most easily made in a laboratory setting. The term “antibody” also refers to any antibody-like molecule that has an antigen binding region, and includes antibody fragments such as Fab′, Fab, F(ab′) 2, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (see, e.g., Antibodies: A Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

Immunodetection methods can be employed to detect levels of protein expression. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev O, 1999; Gulbis and Galand, 1993; De Jager et al., 1993; and Nakamura et al., 1987, each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

In various embodiments, an approach for measuring protein expression levels involves performing a multiplex assay involving the use of oligonucleotide labeled antibody probes that bind to target biomarkers and allow for subsequent quantification of biomarkers. One example of a multiplex assay that involves oligonucleotide labeled antibody probes is the Proximity Extension Assay (PEA) technology (Olink Proteomics). Briefly, a pair of oligonucleotide labeled antibodies bind to a biomarker, wherein the two oligonucleotide sequences are complementary to one another. Thus, only when both antibodies bind to the target biomarker will the oligonucleotide sequences hybridize with one another. Mismatched oligonucleotide sequences (which occurs due to non-specific binding of antibodies or cross-reactivity of antibodies) will not hybridize and therefore, will not result in a readout. Hybridized oligonucleotide sequences undergo nucleic acid extension and amplification, followed by quantification using microfluidic qPCR. The quantified levels correlate to the quantitative expression values of the respective biomarkers.

### Assays for Generating Sequencing Data (e.g., from DNA or RNA)

In various embodiments, performing an assay can include performing one or more of quantitative PCR (qPCR) or digital PCR (dPCR). In various embodiments, performing an assay comprises performing one or more of sequencing of target nucleic acids; hybrid capture; methylation-specific PCR; an assay that generates sequence information; and sequencing a clone library generated from a template immortalized library. In various embodiments, performing an assay can include performing a quantitative PCR assay, such as a quantitative methylation-specific PCR (qMSP) assay. In such embodiments, the methodology generates sequence information in the form of quantitative values that reflect presence or absence of certain target sequences.

Generally, detection of low copies of methylated DNA targets can be challenging. Therefore, qMSP assays can be employed to enrich for bisulfite-converted methylated DNA target sequences in the presence of excess of unmethylated counterpart sequences. The qMSP assays can be real-time PCR assays that utilize sequence-specific primers designed to hybridize with bisulfite-converted target sequences (or sequences flanking or adjacent to target sequences), thereby enabling enrichment of the bisulfite-converted target sequences. In particular embodiments, performing an assay includes performing a qPCR assay, such as a qMSP assay, and further does not include performing sequencing. Performing a qMSP assay without performing sequencing is advantageous in the form of cheaper costs and more rapid turnaround.

In various embodiments, performing an assay can include performing sequencing, such as next-generation sequencing (NGS). NGS, also referred to as massively parallel sequencing, enables the simultaneous sequencing of millions to billions of DNA fragments in a single sequencing run, providing high-throughput analysis of genomic regions of interest at unprecedented scale and resolution. In various embodiments, NGS platforms employ sequencing-by-synthesis chemistry, wherein DNA polymerase incorporates fluorescently labelled nucleotides into growing DNA strands complementary to template molecules immobilized on a solid surface such as a flow cell. During each sequencing cycle, the incorporation of a specific nucleotide (adenine, thymine, cytosine, or guanine) generates a characteristic fluorescent signal that is captured by high-resolution optical imaging systems. Modern NGS instruments utilize flow cell surfaces containing millions of discrete clusters, each representing clonally amplified copies of a single DNA fragment. The sequencing process proceeds through iterative cycles of nucleotide incorporation, imaging, and fluorophore cleavage, with each cycle adding one base to the growing sequence read. Paired-end sequencing configurations sequence both the forward and reverse strands of each DNA fragment, generating two reads per fragment that can improve mapping accuracy and provide additional structural information about the sequenced molecules.

Generally, methylation information refers to methylation statuses for a plurality of genomic sites. In various embodiments, the plurality of genomic sites are previously identified and selected. For example, the plurality of genomic sites may be one or more CpG sites whose differential methylation are informative for determining whether an individual is at risk for a cancer. A CpG site is portion of a genome that has cytosine and guanine separated by only one phosphate group and is often denoted as “5′-C-phosphate-G-3”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIs and features of the genome are referred to herein as “cancer informative CGIs.” The methylation information can be analyzed to generate a prediction for a subject (e.g., whether a sample obtained from the subject includes a presence or absence of a cancer signal).

In various embodiments, performing an assay to generate sequence information for a plurality of genomic sites includes the steps of processing nucleic acids of a sample, enriching the processed nucleic acids for pre-selected genomic sequences (e.g., pre-selected informative CGIs), amplifying the genomic sequences to generate amplicons, and quantifying the amplicons including the genomic sequences (e.g., via sequencing, such as next-generation sequencing, or via quantitative methods such as an ELISA, quantitative PCR, or DNA or RNA-based assay). In various embodiments, performing an assay to generate sequence information for a plurality of genomic sites involves a subset of the previously mentioned steps. For example, enriching the processed nucleic acids can be omitted. Therefore, performing an assay may include processing nucleic acids of a sample, amplifying the pre-selected genomic sequences, and quantifying the amplicons including the genomic sequences.

In various embodiments, the sequence information includes methylation statuses of two or more, three or more, four or more, five or more, six or more, seven or more, eight or more, nine or more, or ten or more genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more genomic sites. In particular embodiments, sequence information of the target nucleic acids and sequence information of the reference nucleic each includes statuses for between 1 and 100 genomic sites, between 2 and 90 genomic sites, between 3 and 80 genomic sites, between 4 and 70 genomic sites, between 5 and 60 genomic sites, between 6 and 50 genomic sites, between 7 and 40 genomic sites, between 8 and 30 genomic sites, between 9 and 20 genomic sites, between 10 and 15 genomic sites. In various embodiments, the plurality of genomic sites include a plurality of CpG islands (CGIs) whose differential methylation status may be indicative of a cancer. Exemplary genomic sites including cancer informative CGIs are described in any one of Tables 4-9.

In various embodiments, the sequence information includes methylation statuses of one or more of Septin9 (UniProt: Q9UHD8), Syndecan 2 (“SDC2”, UniProt: P34741), and Branched Chain Amino Acid Transaminase 1 (“BCAT1”, UniPrto: P54687). In various embodiments, the sequence information includes methylation statuses of two or more of Septin9 (UniProt: Q9UHD8), Syndecan 2 (“SDC2”, UniProt: P34741), and Branched Chain Amino Acid Transaminase 1 (“BCAT1”, UniPrto: P54687). In various embodiments, the sequence information includes methylation statuses of each of Septin9 (UniProt: Q9UHD8), Syndecan 2 (“SDC2”, UniProt: P34741), and Branched Chain Amino Acid Transaminase 1 (“BCAT1”, UniPrto: P54687). In particular embodiments, methods can involve performing a real-time PCR assay (e.g., a qMSP assay) that utilizes sequence-specific primers designed to hybridize with bisulfite-converted sequences (or sequences flanking or adjacent to target sequences) of one or more of Septin9, SDC2, and BCAT1. This enables enrichment of the bisulfite-converted sequences of one or more of Septin9, SDC2, and BCAT1. Septin9 is found on chromosome 17 between positions 77,280,569-77,500,596 (GRCh38/hg38). SDC2 is found on chromosome 8 between positions 96,493,410-96,611,790 (GRCh38/hg38). BCAT1 is found on chromosome 12 between positions chr12: 24,810,024-24,949,556 (GRCh38/hg38). Thus, for performing qMSP, sequence-specific primers can be designed to hybridize with bisulfite-converted sequences within these identified target sequences (or sequences flanking or adjacent to target sequences). For example, for Septin9, sequence-specific primers can be designed to hybridize with bisulfite-converted sequences within target sequences located on chromosome 17 between positions 77,280,569-77,500,596. For example, for SDC2, sequence-specific primers can be designed to hybridize with bisulfite-converted sequences within target sequences located on chromosome 8 between positions 96,493,410-96,611,790. For example, for BCAT1, sequence-specific primers can be designed to hybridize with bisulfite-converted sequences within target sequences located on chromosome 12 between positions chr12: 24,810,024-24,949,556.

A methylated nucleic acid is a nucleic acid having a modification in which a hydrogen atom on the pyrimidine ring of a cytosine base is converted to a methyl group, forming 5-methylcytosine. Methylation can occur at dinucleotides of cytosine and guanine referred to herein as “CpG sites”, which can be a target for enrichment. Methylation of cytosine can occur in cytosines in other sequence contexts, for example, 5′-CHG-3′ and 5′-CHH-3′, where His adenine, cytosine or thymine. Cytosine methylation can also be in the form of 5-hydroxymethylcytosine. Methylation of DNA can include methylation of non-cytosine nucleotides, such as N⁶-methyladenine (6 mA). Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

In certain embodiments, the nucleic acid comprises a CpG site (i.e., cytosine and guanine separated by only one phosphate group). A CpG site is portion of a genome that has cytosine and guanine separated by only one phosphate group and is often denoted as “5′-C-phosphate-G-3”, or “CpG” for short. In certain embodiments, the nucleic acid comprises a CpG island (also referred to as a “CG islands” or “CGI”) or a portion thereof, which is the target for enrichment. Because certain CGIs and certain features of certain CGIs in tumor cells tend to be different from the same CGIs or features of the CGIs in healthy cells, detection of such CGIs can be informative of a cancer. In certain embodiments, the CGI is a “cancer informative CGIs”, which is defined and described in more detail below. In certain embodiments, the CpG is an “informative CpG”, e.g., a “cancer informative CGI”. Such CGIs may have methylation patterns in tumor cells that are different from the methylation patterns in healthy cells. Accordingly, detection of a cancer informative CGI can be informative regarding a subject's risk of developing cancer or can be indicative that the subject has cancer. Exemplary cancer informative CGIs, which can be target sequences as described herein, are identified in, e.g., Table 1 of U.S. Patent Publication 2020/0109456A1, In some embodiments, an analysis of a CGI as contemplated herein may comprise analyzing CpGs within at least a portion of one or more regions in Tables 2 and 3 of WO2022/133315, and Tables 4-9 provided herein. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 4-9. In some embodiments, an analysis of a CGI as contemplated herein may comprise analyzing CpGs within at least a portion of one or more regions in Table 4. In some embodiments, an analysis of a CGI as contemplated herein may comprise analyzing CpGs within at least a portion of one or more regions in Table 5. In some embodiments, an analysis of a CGI as contemplated herein may comprise analyzing CpGs within at least a portion of one or more regions in Table 6. In some embodiments, an analysis of a CGI as contemplated herein may comprise analyzing CpGs within at least a portion of one or more regions in Table 7. In some embodiments, an analysis of a CGI as contemplated herein may comprise analyzing CpGs within at least a portion of one or more regions in Table 8. In some embodiments, an analysis of a CGI as contemplated herein may comprise analyzing CpGs within at least a portion of one or more regions in Table 9.

In certain aspects, the nucleic acids have been treated to convert one or more unmethylated nucleotides (e.g., cytosines) to another nucleotide (a “converted nucleotide”, as used herein, such as a uracil), for example, prior to amplification. Example conversions include bisulfite conversion, enzymatic conversion, or nitrite conversion, further details of which are described herein. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5mC)) is converted to a thymine, which pairs with adenine. In certain embodiments, methylated cytosines are protected from conversion (e.g., deamination) during the conversion step.

In various embodiments, nucleic acids undergo a bisulfite conversion. Bisulfite conversion is performed on DNA by denaturation using high heat, preferential deamination (at an acidic pH) of unmethylated cytosines, which are then converted to uracil by desulfonation (at an alkaline pH). Methylated cytosines remain unchanged on the single-stranded DNA (ssDNA) product.

In some embodiments the methods include treatment of the sample with bisulfite (e.g., sodium bisulfite, potassium bisulfite, ammonium bisulfite, magnesium bisulfite, sodium metabisulfite, potassium metabisulfite, ammonium metabisulfite, magnesium metabisulfite and the like). Unmethylated cytosine is converted to uracil through a three-step process during sodium bisulfite modification. The steps are sulphonation to convert cytosine to cytosine sulphonate, deamination to convert cytosine sulphonate to uracil sulphonate and alkali desulphonation to convert uracil sulphonate to uracil. Conversion on methylated cytosine is much slower and is not observed at significant levels in a 4-16 hour reaction. (See Clark et al., Nucleic Acids Res., 22 (15): 2990-7 (1994).) If the cytosine is methylated it will remain a methylated cytosine. If the cytosine is unmethylated it will be converted to uracil. When the modified strand is copied, for example, through extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated and converted to U. When the double stranded extension product is amplified those Cs that were converted to Us and resulted in incorporation of A in the extended primer will be replaced by Ts during amplification. Those Cs that were not converted (i.e., the methylated Cs) and resulted in the incorporation of G will be replaced by unmethylated Cs during amplification.

In various embodiments, nucleic acids undergo an enzymatic conversion. In certain embodiments, the enzymatic treatment with a cytidine deaminase enzyme is used to convert cytosine to uracil. Enzymatic conversion can include an oxidation step, in which Tet methylcytosine dioxygenase 2 (TET2) catalyzes the oxidation of 5mC to 5hmC to protect methylated cytosines from conversion by subsequent exposure to a cytidine deaminase. Other protection steps known in the art can be used in addition to or in place of oxidation by TET2. After the oxidation step, the nucleic acid is treated with the cytidine deaminase to convert one or more unmethylated cytosines to uracils. As with bisulfite conversion, when the modified strand is copied, a G will be incorporated in the interrogation position (opposite the C being interrogated) if the C was methylated and an A will be incorporated in the interrogation position if the C was unmethylated. When the double stranded extension product is amplified those Cs that were converted to Us and resulted in incorporation of A in the extended primer will be replaced by Ts during amplification. Those Cs that were not modified and resulted in the incorporation of G will remain as C.

In certain embodiments the cytidine deaminase may be APOBEC. In certain embodiments, the cytidine deaminase includes activation induced cytidine deaminase (AID) and apolipoprotein B mRNA editing enzymes, catalytic polypeptide-like (APOBEC). In certain embodiments, the APOBEC enzyme is selected from the human APOBEC family consisting of: APOBEC-1 (Apo1), APOBEC-2 (Apo2), AID, APOBEC-3A, -3B, -3C, -3DE, -3F, -3G, -3H and APOBEC-4 (Apo4). In certain embodiments, the APOBEC enzyme is APOBEC-seq.

In certain embodiments, nitrite treatment is used to deaminate adenine and cytosine. Deamination of an A results in conversion to an inosine (I), which is read by a polymerase as a G, whereas deamination of a methylated A (N⁶-methyladenine (6 mA)) results in a nitrosylated 6 mA (6 mA-NO), which causes the base to be read by a polymerase as an A. Deamination of a C results in conversion to a uracil, which is read by a polymerase as a T, whereas deamination of a N4-methylcytosine (4mC) to 4mC-NO or a 5-methylcytosine (5mC) to a T causes the base to be read by a polymerase as a C or a T, respectively. For 5mC bases, the C to T ratio at the 5mC position is about 40% higher than other cytosine positions, allowing 5mC to be differentiated from C. (See, Li et al. (2022) Genome Biology 23:122.)

In various embodiments, performing the assay includes enriching for specific genomic sequences, such as genomic sequences of pre-selected CGIs. In various embodiments, enrichment of pre-selected CGIs can be accomplished via hybrid capture. Examples of such hybrid capture probe sets include the KAPA HyperPrep Kit and SeqCAP Epi Enrichment System from Roche Diagnostics (Pleasanton, CA). For example, hybrid capture probe sets can be designed to target (e.g., hybridize with) selected genomic sequences, thereby capturing and enriching the selected genomic sequences.

In various embodiments, performing the assay includes a step of nucleic acid amplification. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

Examples of such assays include, but are not limited to performing PCR assays, Real-time PCR assays, Quantitative real-time PCR (qPCR) assays, digital PCR (dPCR), Allele-specific PCR assays, Reverse-transcription PCR assays and reporter assays. For example, given the processed nucleic acids (e.g., bisulfite converted nucleic acids) that are enriched for pre-selected genomic sequences, a PCR assay is performed to amplify the pre-selected genomic sequences to generate amplicons. Here, PCR primers are added to initiate the amplification. In various embodiments, the PCR primers are whole genome primers that enable whole genome amplification. In various embodiments, the PCR primers are gene-specific primers that result in amplification of sequences of specific genes. In various embodiments, the PCR primers are allele-specific primers. For example, allele specific primers can target a genomic sequence corresponding to a pre-selected CGI, such that performing nucleic acid amplification results in amplification of the genomic sequence of the pre-selected CGI.

In various embodiments, performing the assay includes quantifying the nucleic acids including the pre-selected genomic sequences (e.g., informative CGIs). In some embodiments, quantifying the nucleic acids to generate sequence information comprises performing an enzyme-linked immunosorbent assay (ELISA). In some embodiments, quantifying the nucleic acids to generate sequence information comprises performing quantitative PCR (qPCR) or digital PCR (dPCR). Therefore, the number of methylated, unmethylated, or partially methylated pre-selected genomic sequences can be quantified.

In various embodiments, quantifying the nucleic acids comprises sequencing the nucleic acids including the pre-selected genomic sequences. Thus, the sequenced reads can be aligned to a reference library and methylation sequence information including methylation statuses of the informative CGIs can be determined. Therefore, the number of methylated, unmethylated, or partially methylated pre-selected genomic sequences can be quantified via the sequenced reads.

### Assays for Data of Extracellular Vesicles

In various embodiments, performing an assay can include generating data from extracellular vesicles (EVs), examples of which include RNA expression levels, where the RNA is derived from EVs. EVs include exosomes, microvesicles, and/or apoptotic bodies. In various embodiments, performing an assay involves isolating and/or enriching for extracellular vesicles (EVs). In many scenarios, EVs are present at small quantities in the test sample and therefore, isolating and/or enriching for the EVs is a valuable step. Furthermore, as EVs are typically small in diameter (e.g., 30 nm up to 1000 nm), the amount of nucleic acids contained within a single EV can be small. Therefore, isolating and/or enriching for EVs would enable access to the contents of the EVs (e.g., the exoRNA markers).

In various embodiments, EVs are isolated and/or enriched based on properties of the EVs, examples of which include the size, density, electrical properties, and/or the morphology of the EVs. In various embodiments, isolating and/or enriching for EVs involves using precipitation and spin columns, filtration techniques, ultracentrifugation (e.g., at speeds greater than 100,000g), size exclusion chromatography (e.g., Exo-spin, qEV), or affinity selection. Further details for isolating and enriching for EVs are described in Zhao et al., Isolation and analysis methods of extracellular vesicles (EVs), Extracell Vesicles Circ Nucl Acids. 2021; 2 (1): 80-103, the entirety of which is incorporated by reference in its entirety.

In various embodiments, performing assay involves detecting the cargo within the EVs. In various embodiments, the cargo within the EVs include any of proteins, lipids, carbohydrates, cytokines, and nucleic acids. In particular embodiments, performing the extracellular vesicle assay 120 involves detecting RNA molecules in the EVs. Detecting the cargo within the EVs can involve performing any one of Western blotting, enzyme-linked immunosorbent assay (ELISA), polymerase chain reaction (PCR), real-time PCR (RT-PCR), real-time quantitative PCR (RT-qPCR), and next generation sequencing (NGS).

In particular embodiments, detecting the cargo within the EVs involves performing nucleic acid enrichment. Nucleic acids, such as RNA molecules, can be amplified using paired primers flanking a region of interest. Here, the region of interest can include a region of a gene, such as a RNA marker. Example regions of interest can be any genomic region of a RNA marker identified in any one of Tables 1-3. The term “primer,” as used herein, is meant to encompass any nucleic acid that is capable of priming the synthesis of a nucleic acid in a template-dependent process. Typically, primers are oligonucleotides from ten to twenty and/or thirty base pairs in length, but longer sequences can be employed. Primers may be provided in double-stranded and/or single-stranded form.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864. In various embodiments, nucleic acid enrichment involves multiplex-PCR. Whereas standard PCR usually uses one pair of primers to amplify a specific sequence, multiplex-PCR (MPCR) uses multiple pairs of primers to amplify many sequences simultaneously.

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

### Assays for Metabolic Data

In various embodiments, performing an assay involves generating metabolic data (e.g., data of levels of one or more metabolites). Generally, the metabolic data provide a view of the physiology of a cell at a particular time, such as the levels of metabolites in the cell or produced by the cell at the particular time. Metabolic data may be represented as a metabolome e.g., as a complete set of metabolites. Examples of metabolic data include detected metabolite levels expressed by cells, a ratio of the levels of two associated metabolites (e.g., ratio of levels of a first metabolite and a second metabolite, the first metabolite being a precursor of the second metabolite), or a ratio of the level of a metabolite in relation to a reference value (e.g., a reference metabolite level in healthy individuals). In various embodiments, these example metabolite data can serve as features of the machine learning model.

In various embodiments, a metabolite is less than 1.5 kDa in size. Examples of metabolites include oxygen, carbon dioxide, glucose, insulin, lactate, glutamine, glutamate, lipoproteins, albumin, fatty acids, ATP, and NADH associated molecules (e.g., NAD, NADP, NADPH). Additional example metabolites can be found in publicly available databases such as METLIN or the Human Metabolome Database (HMDB).

In various embodiments, detection of example metabolites can use commercially available kits that are designed to facilitate the determination of quantitative levels of different metabolites. Examples of commercially available kits include ABCAM assays for measuring oxygen consumption, glycolysis, fatty acid metabolism, ATP, NADH, and associated molecules, PROMEGA assays for NAD, NADP, NADH, and NADPH assays, Metabolite assays (glucose, lactate, glutamine, glutamate), and Thermo Fisher Scientific assays such as ATP determination kit, Amplex™ assay kits, ThioTracker™ assays, or Vybrant™ Cell Metabolic Assay kit.

Generally, the kits involve adding one or more reagents to a sample including metabolites, the one or more reagents able to bind or interact with a target metabolite. The interaction between a reagent and a target metabolite can be detected using a variety of detection methods including flow cytometry, fluorescence microscopy, microplate (e.g., bioluminescence, chemiluminescence, or fluorescence reader), or a spectrometer. In various embodiments, the detected intensity level is a direct or indirect readout for the concentration of the target metabolite in the sample.

In various embodiments, metabolites can be detected using metabolite detection techniques such as nuclear magnetic resonance (NMR), mass spectrometry (MS), or Infrared spectroscopy (IS). Generally, such methods involve the use of isotopes for detecting a metabolite. Methods for detecting target metabolites using isotopes are described in U.S. Pat. No. 6,849,396, which is hereby incorporated by reference in its entirety.

For mass spectrometry, analysis of the following different classes of metabolites can be found in: (1) lipids (see, e.g., Fenselau, C., “Mass Spectrometry for Characterization of Microorganisms”, ACS Symp. Ser., 541:1-7 (1994)); (2) volatile metabolite (see, e.g., Lauritsen, F. R. and Lloyd, D., “Direct Detection of Volatile Metabolites Produced by Microorganisms,” ACS Sympl Ser., 541:91-106 (1994)); (3) carbohydrates (see, e.g., Fox, A. and Black, G. E., “Identification and Detection of Carbohydrate Markers for Bacteria”, ACS Symp. Ser. 541:107-131 (1994); (4) nucleic acids (see, e.g., Edmonds, C. G., et al., “Ribonucleic acid modifications in microorganisms”, ACS Symp. Ser., 541:147-158 (1994); and (5) proteins (see, e.g., Vorm, O. et al., “Improved Resolution and Very High Sensitivity in MALDI TOF of Matrix Surfaces made by Fast Evaporation,” Anal. Chem. 66:3281-3287 (1994); and Vorm, O. and Mann, M., “Improved Mass Accuracy in Matrix-Assisted Laser Desorption/Ionization Time-of-Flight Mass Spectrometry of Peptides”, J. Am. Soc. Mass. Spectrom. 5:955-958 (1994)). Each of these are hereby incorporated by reference in their entirety. Furthermore, IR and NMR methods for conducting isotopic analyses are discussed, for example, in U.S. Pat. No. 5,317,156; Klein, P. et al., J. Pediatric Gastroenterology and Nutrition 4:9-19 (1985); Klein, P., et al., Analytical Chemistry Symposium Series 11:347-352 (1982), each of which is hereby incorporated by reference in its entirety.

In various embodiments, metabolites are detected from purified/separated samples, thereby removing other components (e.g., cellular debris) that may impact the sensitivity and/or specificity of the detection. For example, samples may be purified using electrophoresis or high performance liquid chromatography. Therefore, the purified samples can be analyzed using NMR, MS, or IS to detect metabolite concentrations.

### Assays for Lipidomic Data

In various embodiments, performing an assay involves generating lipidomic data (e.g., data of levels of one or more lipids). In various embodiments, the one or more lipids include, without limitation, any of cholesterol, triglycerides, apolipoproteins, fatty acids, or sterols. In various embodiments, performing an assay to generate lipidomic data comprises performing any one of electrophoresis (e.g., gradient gel electrophoresis and/or ion mobility), centrifugation (e.g., ultracentrifugation), nuclear magnetic resonance (NMR), precipitation methods, immunoassays, mass spectrometry (MS) (e.g., any of MALDI-MS, secondary ion MS, or electrospray ionization MS), Raman-based technologies, or fluorescent-based technologies. Further details for performing assays to generate lipidomic data are described in Yao M, et al., Analytical Techniques for Single-Cell Biochemical Assays of Lipids. Annu Rev Biomed Eng. 2023 Jun. 8; 25:281-309, which is incorporated by reference in its entirety.

### Computer Implementation

The methods disclosed herein, including the methods for prioritizing healthcare resources in cancer care, are, in some embodiments, performed on one or more computers. In various embodiments, the methods for prioritizing healthcare resources in cancer care can be implemented in hardware or software, or a combination of both. In one embodiment, a machine-readable storage medium is provided, the medium comprising a data storage material encoded with machine readable data which, when using a machine programmed with instructions for using said data, is capable of displaying data and results of analysis. Such data can be used for a variety of purposes, such as for categorizing subjects and/or determining a frequency of healthcare utilization for subjects based on the categorization. The disclosed methods can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

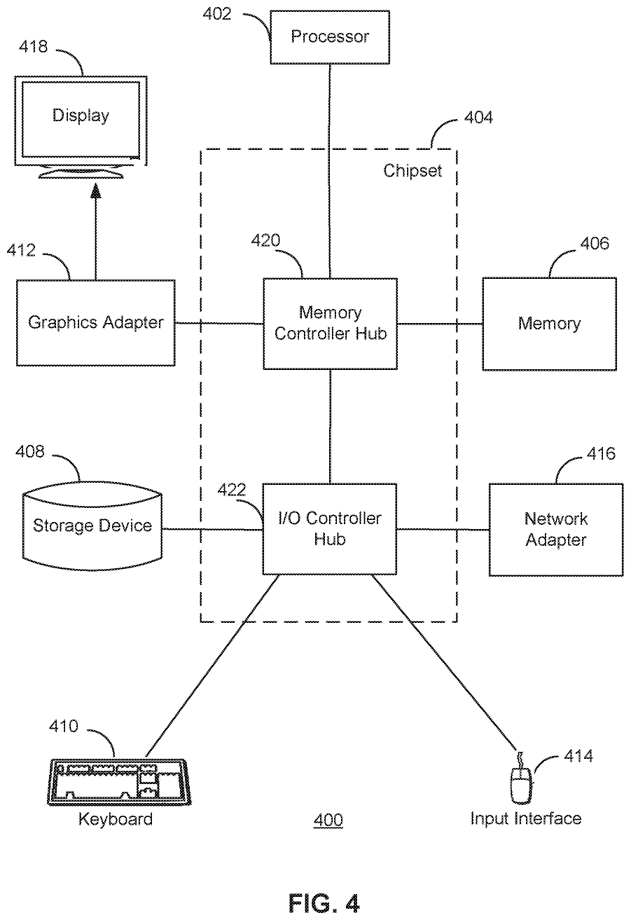

FIG. 4 illustrates an example computer for implementing the entities shown in FIGS. 1A-1B, 2, and 3.

The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input device 414 is a touch-screen interface, a mouse, track ball, or some combination thereof, and is used to input data into the computer 400. The keyboard 410 may be another device for inputting data into the computer 400. In some embodiments, the computer 400 may be configured to receive input (e.g., commands) from the input device 414 via gestures from the user. The graphics adapter 412 displays images and other information on the display 418. The network adapter 416 couples the computer 400 to one or more computer networks.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

In various embodiments, methods disclosed herein can be performed on a single computer 400 or multiple computers 400 communicating with each other through a network such as in a server farm. In various embodiments, the computers 400 can lack some of the components described above, such as graphics adapters 412, and displays 418.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods disclosed herein, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Additional Embodiments

Disclosed herein is a method for prioritizing healthcare resources for patient care, the method comprising; (a) obtaining a sample from a subject, wherein the subject was previously identified as at risk of a cancer through a prior screen; (b) processing the sample to generate data of at least one or more analytes; (c) stratifying the subject into a risk category by analyzing the data of at least one or more analytes; and (d) assigning the subject a frequency of healthcare utilization according to the stratified risk category of the subject. In certain embodiments, the sample may contain multiple analytes, for example protein analytes and nucleic acid analytes.

In various embodiments, the at least one or more analytes comprise two different nucleic acid analytes or two protein analytes. In various embodiments, the two or more analytes comprise a protein analyte and a nucleic acid analyte. In various embodiments, the two or more analytes comprise a protein analyte, a DNA analyte, and a RNA analyte.

In various embodiments, the risk category is one of a high risk, intermediate risk, or low risk. In various embodiments, the frequency of healthcare utilization determined for the subject in the high risk category is greater than the frequency of healthcare utilization determined for a subject in the intermediate risk category and/or the low risk category. In various embodiments, the data further comprises data of one or more lipids or data from one or more metabolites. In various embodiments, processing the sample comprises performing one or more of a sequencing assay of nucleic acids, a protein assay, or a metabolite detection assay. In various embodiments, performing the sequencing assay comprises sequencing nucleic acids derived from exosomes, sequencing nucleic acids derived from DNA, or sequencing nucleic acids derived from RNA. In various embodiments, performing the sequencing assay comprises sequencing converted nucleic acids derived from cell-free DNA (cfDNA). In various embodiments, the converted nucleic acids comprise bisulfite converted nucleic acids derived from cfDNA.

In various embodiments, performing the sequencing assay comprises performing hybrid capture and/or polymerase chain reaction (PCR), optionally wherein the PCR is a methylation-specific PCR. In various embodiments, the protein assay comprises a protein immunoassay, an enzyme linked immunosorbent assay (ELISA), radioimmunoassay (RIA), mass spectrometry (MS), or an antibody-based proximity binding assay. In various embodiments, the metabolite detection assay comprises nuclear magnetic resonance (NMR), mass spectrometry (MS), or Infrared spectroscopy (IS).

In various embodiments, the data of one or more nucleic acid analytes comprises sequencing data of DNA. In various embodiments, the sequencing data of DNA comprises methylation statuses of a plurality of genomic sites of the DNA. In various embodiments, the plurality of genomic sites of the DNA comprises one or more genomic regions shown in any of Tables 4-9. In various embodiments, the plurality of genomic sites of the DNA comprises one or more genomic regions shown in any of Tables 8 or 9. In various embodiments, the healthcare utilization comprises a medical procedure, a future visit to a hospital, the use of a specialist, a diagnostic testing, a diagnostic imaging, a biopsy, a medical appointment, and/or a prophylactic medication. In various embodiments, the medical procedure comprises a colonoscopy. In various embodiments, diagnostic imaging comprises one or more of an ultrasound scan, a MRI scan, or a CT scan. In various embodiments, the prior screen comprises a family history evaluation, a life style evaluation, an age group, a demographic group, a prior cancer screening test and/or a prior cancer diagnostic test.

In various embodiments, the prior screen comprises COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, imaware Prostate Cancer screening test, a PSA blood test, a mammogram, a colonoscopy, a Pap smear, am HPV test, a biopsy, a hereditary cancer screen (for example the Natera Empower test, Guardant Hereditary cancer test, Myriad Genetics hereditary cancer test or Labcorp hereditary cancer test), low-dose computed tomography (LDCT), a prenuvo cancer scan, or the Grail Galleri multi-cancer screening.

In various embodiments, the cancer is any of gastrointestinal (GI) cancer, acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In various embodiments, the GI cancer is colorectal cancer, esophageal cancer, stomach cancer, gallbladder cancer, liver cancer, pancreatic cancer, small intestinal risk, rectal cancer, appendix cancer, anal cancer, and/or colon cancer.

Additionally disclosed herein is a method for prioritizing healthcare resources for patient care, the method comprising; (a) obtaining or having obtained data of at least two or more analytes generated from a multianalyte sample obtained from a subject previously identified as at risk of a cancer through a prior screen; (b) stratifying the subject into a risk category by analyzing the data of at least the two or more analytes; and (c) assigning the subject a frequency of healthcare utilization according to the stratified risk category of the subject.

In various embodiments, the two or more analytes comprise two different nucleic acid analytes. In various embodiments, the two or more analytes comprise a protein analyte and a nucleic acid analyte. In various embodiments, the two or more analytes comprise a protein analyte, a DNA analyte, and a RNA analyte.

In various embodiments, the risk category is one of a high risk, intermediate risk, or low risk. In various embodiments, the frequency of healthcare utilization determined for the subject in the high risk category is greater than the frequency of healthcare utilization determined for a subject in the intermediate risk category and/or the low risk category. In various embodiments, the data further comprises data of one or more lipids or data from one or more metabolites. In various embodiments, the data of one or more nucleic acid analytes comprises sequencing data of DNA. In various embodiments, the sequencing data of DNA comprises methylation statuses of a plurality of genomic sites of the DNA. In various embodiments, the healthcare utilization comprises a medical procedure, a future visit to a hospital, the use of a specialist, a diagnostic testing, a diagnostic imaging, a biopsy, a medical appointment, and/or a prophylactic medication. In various embodiments, the medical procedure comprises a colonoscopy. In various embodiments, diagnostic imaging comprises one or more of an ultrasound scan, a MRI scan, or a CT scan.

In various embodiments, the prior screen comprises a family history evaluation, a life style evaluation, an age group, a demographic group, and/or a prior cancer diagnostic test. In various embodiments, the cancer diagnostic testing comprises a colonoscopy, a fecal immunochemical test, an ultrasound scan, a MRI scan, a CT scan, COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, or imaware Prostate Cancer screening test. In various embodiments, the cancer is any of gastrointestinal (GI) cancer, acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In various embodiments, the GI cancer is colorectal cancer, esophageal cancer, stomach cancer, gallbladder cancer, liver cancer, pancreatic cancer, small intestinal risk, rectal cancer, appendix cancer, anal cancer, and/or colon cancer.

Additionally disclosed herein is a method for prioritizing patient care for one or more subjects of a patient population for prioritization of healthcare resources, the method comprising: for each of the one or more subjects of the patient population: (a) obtaining a multianalyte sample from the subject, wherein the subject was previously identified as at risk of a cancer through a prior screen; (b) processing the multianalyte sample to generate data of at least one or more protein analytes and one or more nucleic acid analytes; (c) stratifying the subject into a risk category by analyzing the data of at least one or more protein analytes and one or more nucleic acid analytes; and (d) assigning the subject a frequency of healthcare utilization according to the stratified risk category of the subject.

In various embodiments, the two or more analytes comprise two different nucleic acid analytes. In various embodiments, the two or more analytes comprise a protein analyte and a nucleic acid analyte. In various embodiments, the two or more analytes comprise a protein analyte, a DNA analyte, and a RNA analyte.

In various embodiments, the risk category is one of a high risk, intermediate risk, or low risk. In various embodiments, the frequency of healthcare utilization determined for the subject in the high risk category is greater than the frequency of healthcare utilization determined for a subject in the intermediate risk category and/or the low risk category. In various embodiments, the data further comprises data of one or more lipids or data from one or more metabolites.

In various embodiments, processing the multianalyte sample comprises performing one or more of a sequencing assay of nucleic acids, a protein assay, or a metabolite detection assay. In various embodiments, performing the sequencing assay comprises sequencing nucleic acids derived from exosomes, sequencing nucleic acids derived from DNA, or sequencing nucleic acids derived from RNA. In various embodiments, performing the sequencing assay comprises sequencing converted nucleic acids derived from cell-free DNA (cfDNA). In various embodiments, the converted nucleic acids comprise bisulfite converted nucleic acids derived from cfDNA. In various embodiments, performing the sequencing assay comprises performing hybrid capture and/or polymerase chain reaction (PCR), optionally wherein the PCR is a methylation-specific PCR. In various embodiments, the protein assay comprises a protein immunoassay, an enzyme linked immunosorbent assay (ELISA), radioimmunoassay (RIA), mass spectrometry (MS), or an antibody-based proximity binding assay. In various embodiments, the metabolite detection assay comprises nuclear magnetic resonance (NMR), mass spectrometry (MS), or Infrared spectroscopy (IS). In various embodiments, the data of one or more nucleic acid analytes comprises sequencing data of DNA. In various embodiments, the sequencing data of DNA comprises methylation statuses of a plurality of genomic sites of the DNA. In various embodiments, the healthcare utilization comprises the use of a specialist, a diagnostic testing, a diagnostic imaging, a biopsy, a medical appointment, and/or a prophylactic medication.

In various embodiments, the prior screen comprises a family history evaluation, a life style evaluation, an age group, a demographic group, and/or a prior cancer test. In various embodiments, the cancer testing comprises a colonoscopy, a fecal immunochemical test, an ultrasound scan, a MRI scan, a CT scan, COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, or imaware Prostate Cancer screening test. In various embodiments, the cancer is any of gastrointestinal (GI) cancer, acute lymphoblastic leukemia, acute myeloid leukemia, adrenocortical carcinoma, soft tissue sarcoma, lymphoma, anal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, uterine cancer, head and neck cancer, eye cancer, fallopian tube cancer, gallbladder cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, testicular cancer, throat cancer, thymoma and thymic carcinoma, thyroid cancer, urethral cancer, uterine cancer, vaginal cancer, and vulvar cancer. In various embodiments, the GI cancer is colorectal cancer, esophageal cancer, stomach cancer, gallbladder cancer, liver cancer, pancreatic cancer, small intestinal risk, rectal cancer, appendix cancer, anal cancer, and/or colon cancer.

Additionally disclosed herein is a non-transitory computer readable medium comprising instructions that, when executed by a processor, cause the processor to perform methods disclosed herein. Additionally disclosed herein is a computer system comprising: a processor; and a non-transitory computer readable medium comprising instructions that, when executed by a processor, cause the processor to perform methods disclosed herein.

### Examples

### Example 1: Prioritization of Healthcare Resources for Early Detection of Cancer

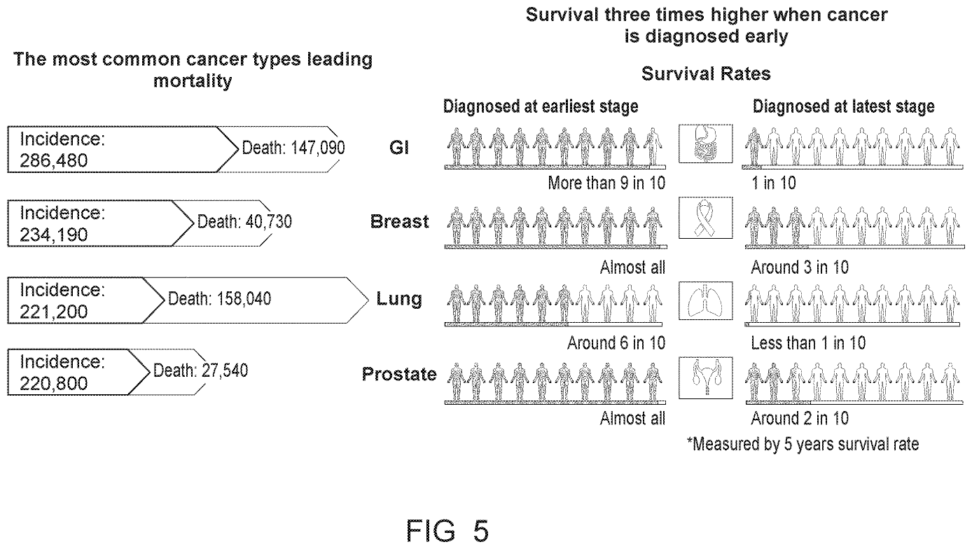

FIG. 5 depicts incidence and death rates of common cancers as well as improved survival of subjects for those common cancers depending on when the cancer was diagnosed. Generally, a large proportion of subjects diagnosed with common cancers (e.g., gastrointestinal (GI), breast, lung, or prostate cancer) lead to mortality. However, there is a significant difference in survival depending on whether the diagnosis occurs at early or late stage. Specifically, survival is three times higher when cancer is diagnosed early. Thus, there is a drive for technologies that are capable of detecting cancer at its earliest stages.

As efforts focus on early detection of cancer, more healthcare resources are dedicated to those processes. For example, the US Preventive Services Task Force (USPSTF) now recommends that adults begin screening for colorectal cancer at an earlier age of 45 (as opposed to 50), which places additional burden on the healthcare system for performing such tests. The high demand for colonoscopies due to the updated guidance has led to underserved patient populations due to the difficulty in scheduling and/or obtaining the test.

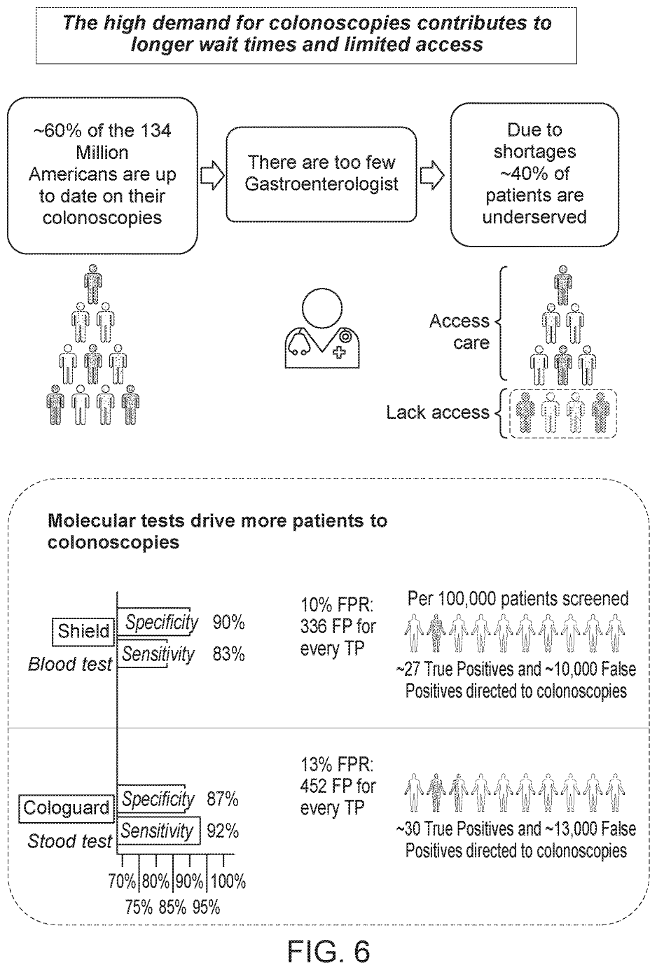

FIG. 6 depicts the burden on healthcare utilization, specifically colonoscopies, for early detection of cancer. Molecular diagnostic tests often suffer from inaccuracies (resulting in high false positive rates). Thus, these subjects who have been falsely identified as positive for cancer undergo unneeded colonoscopies, which only serve to further burden the healthcare system.

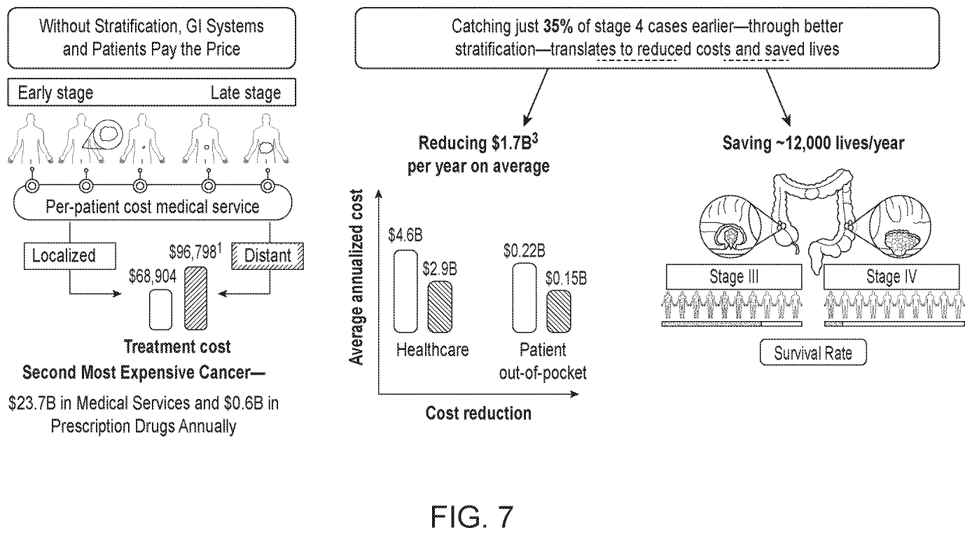

FIG. 7 depicts the value of effective patient stratification which results in reduced costs and increased lives saved. Specifically, without stratification, GI systems and patients may increase the costs associated with the testing. By implementing patient stratification, this enables detection of earlier stages of cancer (prior to Stage 4 cancer) while reducing costs (e.g., reducing $1.7B per year) and saving ~12,000 lives/year.

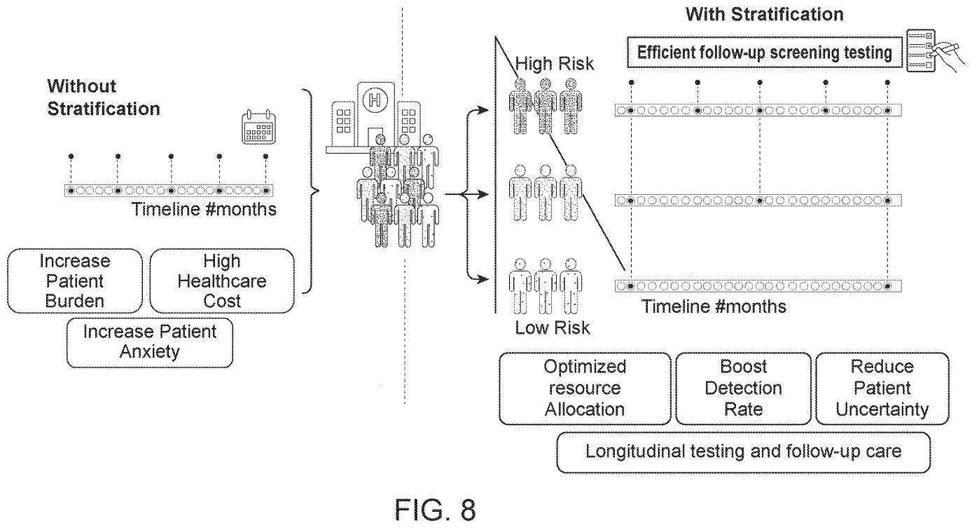

FIG. 8 is a diagrammatic depiction of stratification of subjects which enables optimized allocation of healthcare resources. For example, subjects that undergo testing (e.g., through analysis of a multi-analyte sample obtained from the subjects) undergo stratification into various risk categories (e.g., high risk, low risk, and intermediate risk). As shown in FIG. 8, the frequency of longitudinal testing and follow-up care are dependent on the risk categorization of the subjects. High risk subjects undergo longitudinal testing and follow up at frequencies that are higher than frequencies of intermediate and low risk subjects. Intermediate subjects undergo longitudinal testing and follow up at frequencies that are higher than frequencies of low risk subjects.

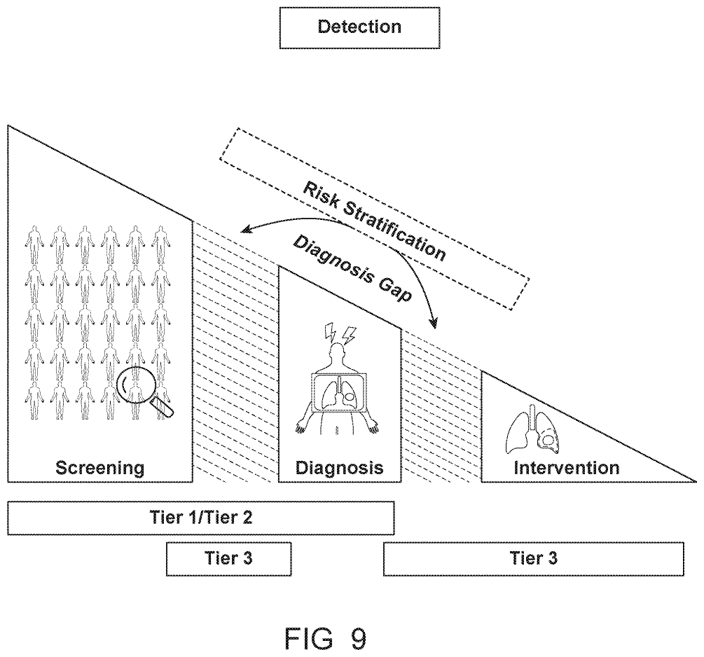

FIG. 9 depicts the risk stratification step that is performed either between Tier 1 and Tier 2, or during tier 3 monitoring stages. As referred to herein, the “Tier 1” test refers to a screen in which large populations of subjects are screened to remove large proportions of subjects who are negative for cancer. The “Tier 2” involves a more complex and/or more expensive test in comparison to the Tier 1 test and is applied for subjects who are not eliminated as a result of the Tier 1 test. The Tier 2 test is therefore applied for a significantly smaller number of subjects in comparison to the Tier 1 test. The “Tier 3” test refers to longitudinal tracking of subjects following the Tier 2 test. Thus, the Tier 3 test refers to continuous tracking of subjects to determine development or progression of cancer in subjects.

Risk stratification is performed either after Tier 1 (to stratify subjects prior to the Tier 2 test) or after Tier 2 test (to stratify subjects prior to Tier 3 longitudinal tracking). Thus, subjects that are stratified into a higher risk category undergo more frequent longitudinal tracking and follow up throughout Tier 3 testing.

### Example 2: Example Analysis of Multianalyte Sample for Prioritization of Healthcare Resources

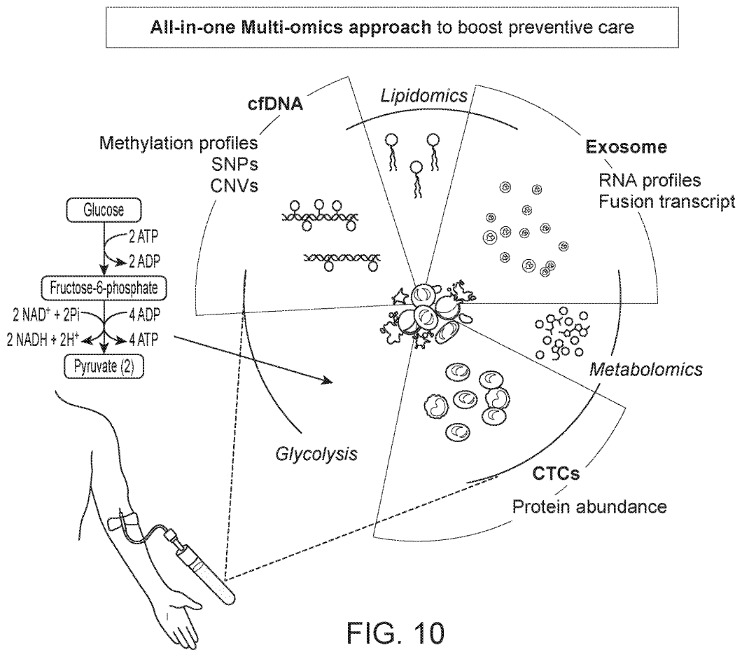

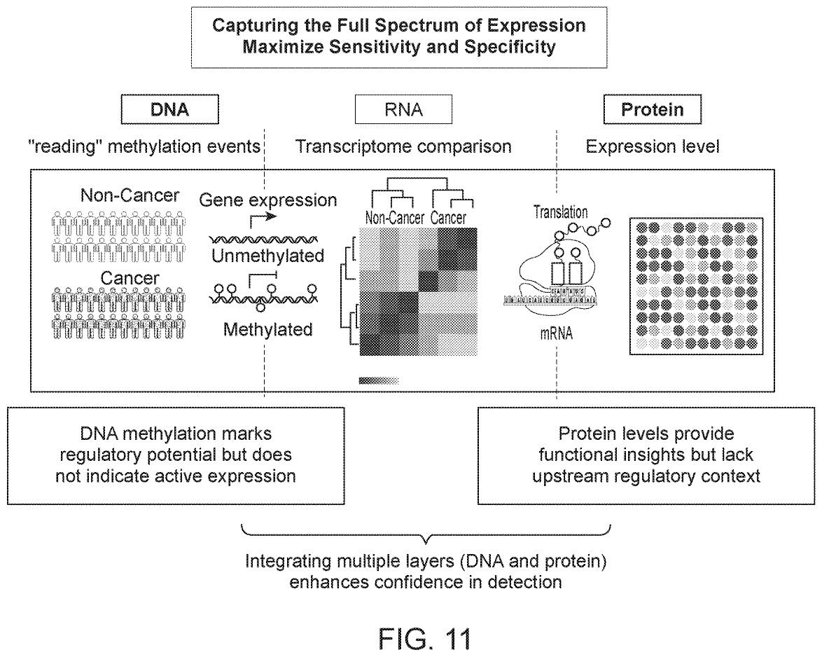

FIG. 10 depicts an example analysis involving a multi-omic approach (e.g., including analysis of cfDNA, lipidomics, exosomal RNA, metabolomics, and/or proteins from circulating tumor cells (CTCs). The multi-omic approach includes additive information from combinations of the inputs. For example, referring to FIG. 11, it depicts the additive benefit of analyzing DNA and protein (in addition to RNA). DNA information (e.g., DNA methylation information) provides insight to regulatory potential, but does not indicate active expression of genes. Therefore, protein expression levels, which provide functional insight to actively expressed genes, provide additional insight when combined with DNA information.

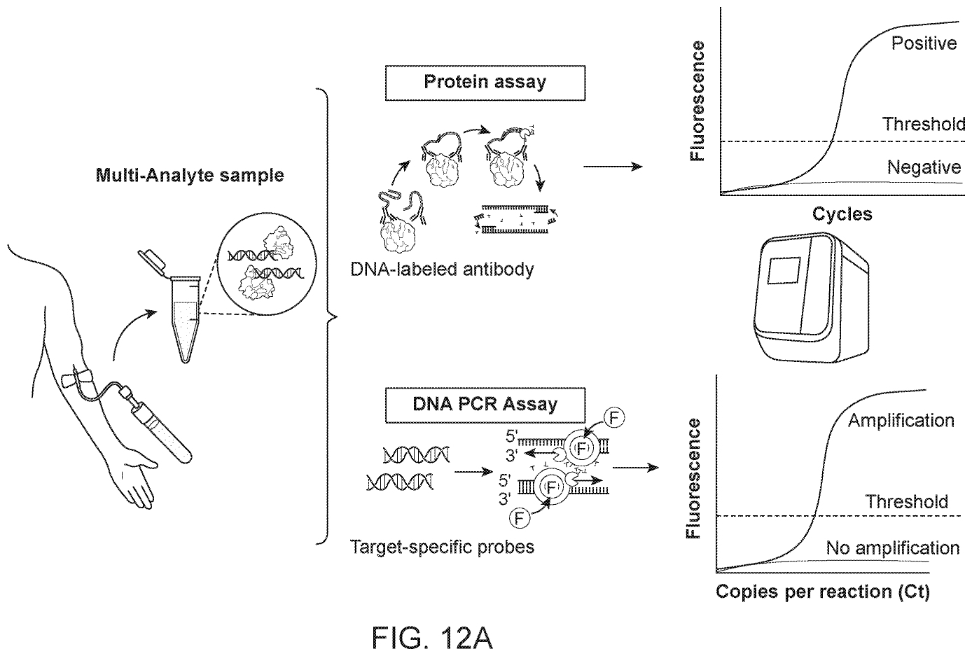

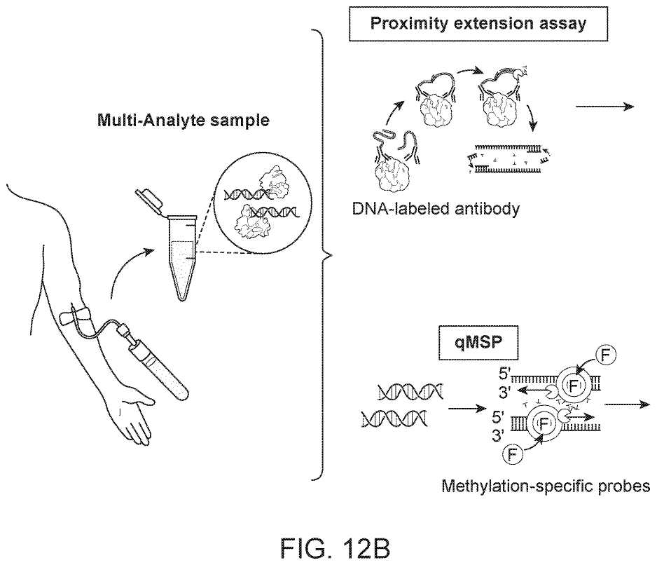

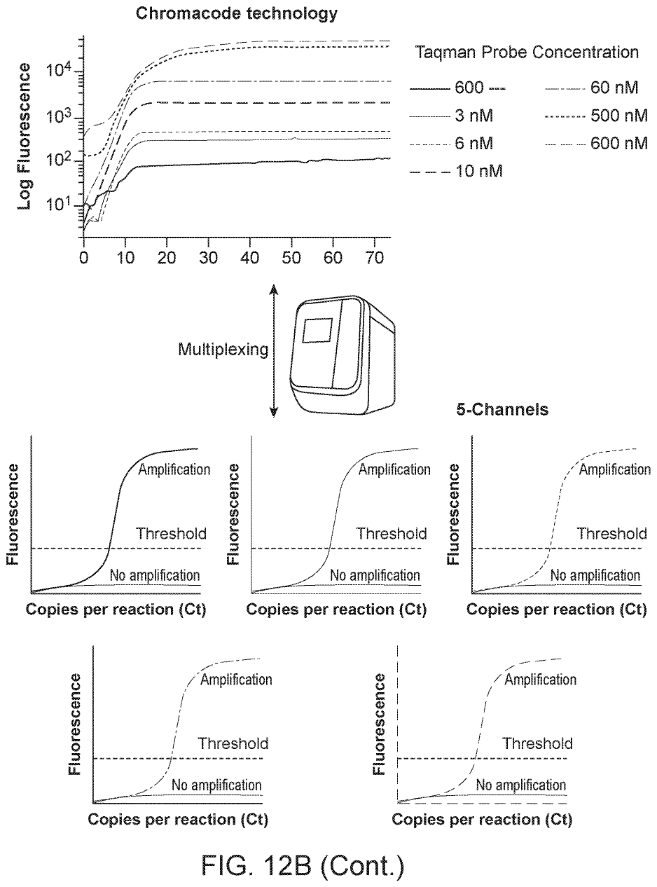

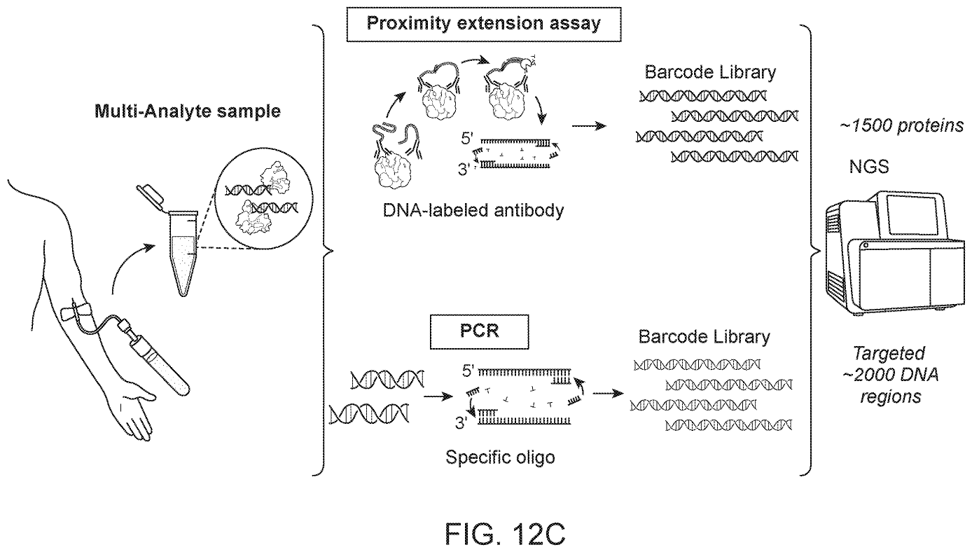

FIGS. 12A-12C depict exemplary methodologies for analyzing multi-analyte samples. For example, FIG. 12A shows an exemplary method involving a protein immunoassay for detecting protein expression levels and a DNA PCR assay for detecting DNA information. As another example, FIG. 12B shows an exemplary method involving a proximity extension assay for detecting protein expression levels and a quantitative multiplex methylation specific PCR (QMSP) for detecting DNA information. As another example, FIG. 12C shows an exemplary method involving a proximity extension assay for detecting protein expression levels and PCR for detecting DNA information. Each of the protein expression levels and DNA information are read out via next generation sequencing (NGS) techniques.

### Example 3: Example Analysis of Multianalyte Sample for Stratifying Breast Cancer Patients

Multianalyte liquid biopsy samples are obtained from subjects (e.g., breast cancer subjects or subjects suspected of having breast cancer). Various analytes including cfDNA, RNA, and/or protein analytes are analyzed. Specifically, a machine learning model is deployed to analyze the various analytes from the multianalyte liquid biopsy samples. The results of the machine learning model are coupled with imaging results captured from the subjects. For example, subjects undergo a breast mammography to determine likely risk of breast cancer. Thus the results of the machine learning model are coupled with the breast mammography for risk stratification. Subjects categorized in the high risk category (in view of the machine learning model results and breast mammography results) undergo more frequent healthcare utilization (e.g., more frequent follow up tests).

### Example 4: Example Multianalytes for Liquid Biopsies

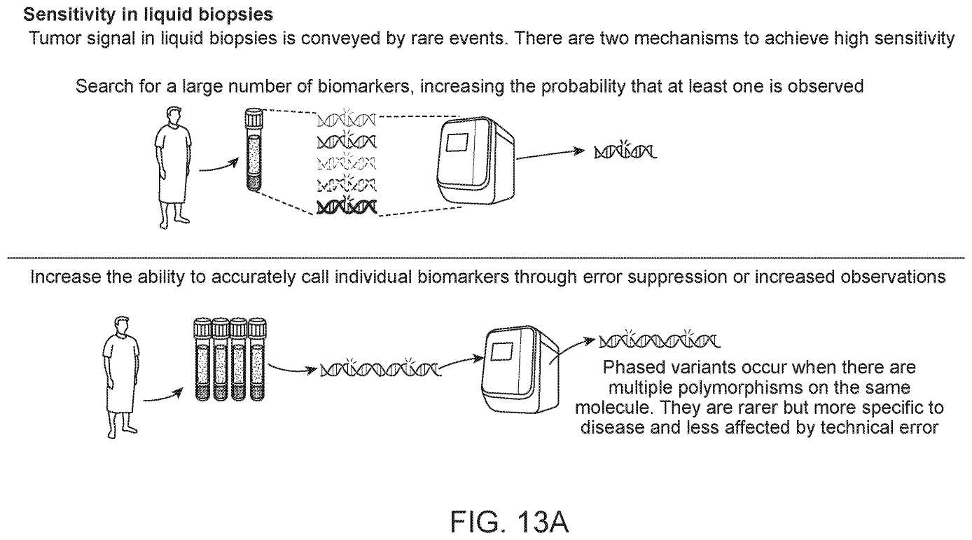

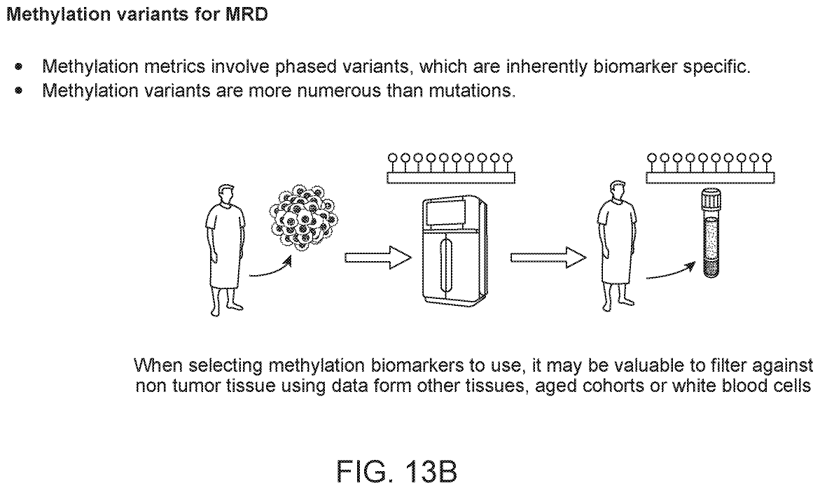

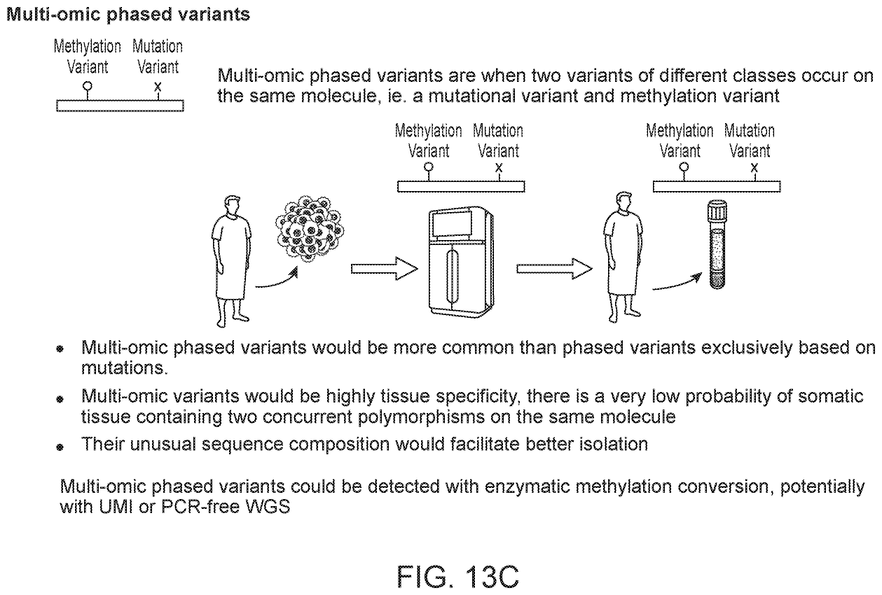

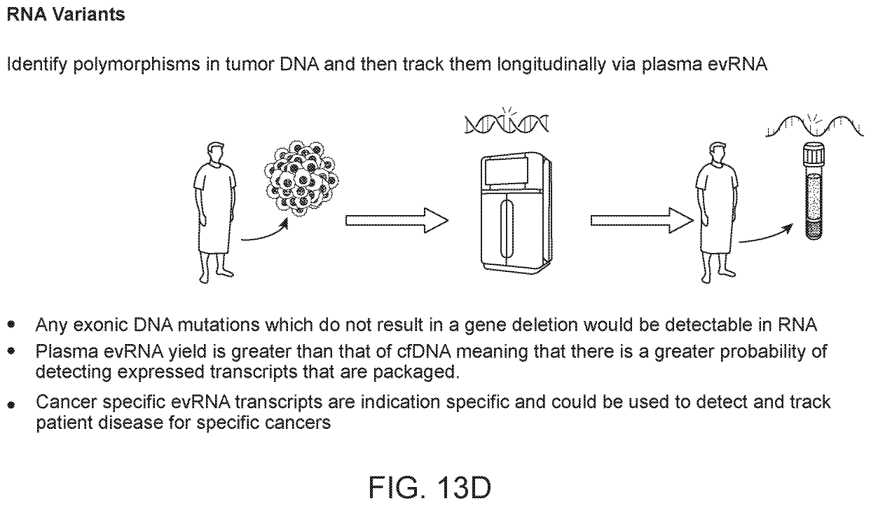

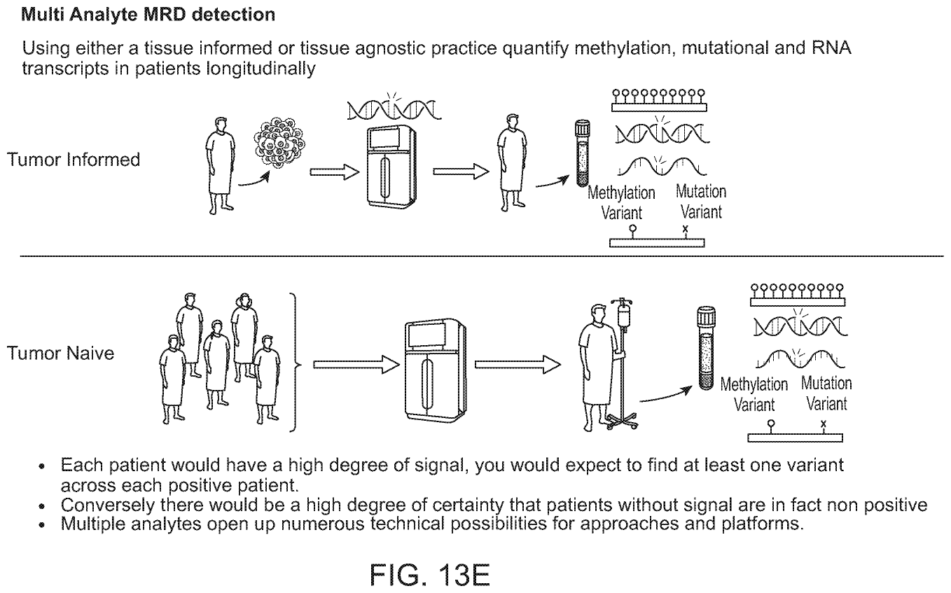

FIGS. 13A-13E depict exemplary multianalytes in liquid biopsies. FIG. 13A depicts two strategies for increasing sensitivity in liquid biopsies. Specifically, one particular method, achieved through use of phased variants, increases the ability to accurately call individual biomarkers through e.g., increased observations.

FIG. 13B depicts an example of a methylation variant, which can be used for purposes of a phased variant. Methylation variants, which refer to particular CpG methylation sites, are significantly more numerous in comparison to mutations (e.g., single nucleotide variants). FIG. 13C depicts example multi-omic phased variants. A multi-omic phased variant includes at least two variants of two different classes that occur on the same molecule. As shown in FIG. 13C, an example multi-omic phased variant includes a methylation variant and a mutation variant (e.g., single nucleotide variant) located on the same molecule. Such multi-omic phased variants that include both a methylation variant and mutation variant would be more commonly observed in comparison to phased variants exclusively based on mutation variants.

FIG. 13C depicts an additional variant derived from a different analytes, specifically from RNA. Here, RNA variants can be detected from extravesicular RNA (evRNA). evRNA can be used to longitudinally track various cancers across multiple samples obtained from subjects.

FIG. 13D shows the combination of a multianalyte, multi-omic phased variant methodology for e.g., minimal residual disease (MRD). The methodology includes either a tumor informed or tumor naïve analysis. Here, the combination of 1) methylation variant, 2) mutation variant, and 3) RNA variant is useful as a multi-omic phased variant. Such a multi-omic phased variant would increase the overall sensitivity of the test.

### Example 5: Analysis of Methylation Pattern for Liquid Biopsies

To provide a high sensitivity, low cost method for identifying informative methylation patterns useful for cancer detection and localization in blood, a discovery set of 386 cancer tissue samples (including over 20 cancer types), and 508 cfDNA samples (including 180 cancer and 328 non cancer samples), was analyzed. For defining informative regions, methylation signal at every CpG was quantified and analyzed by two approaches.

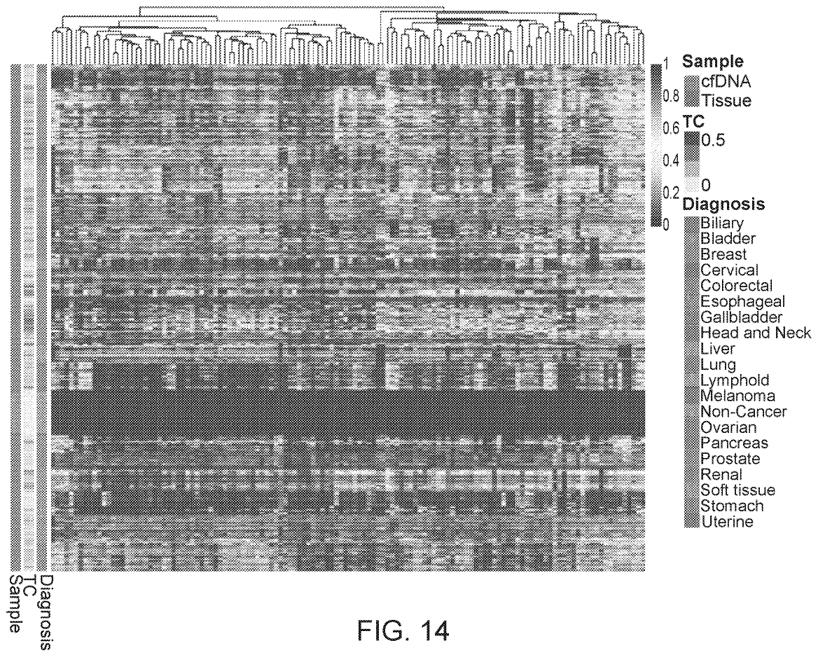

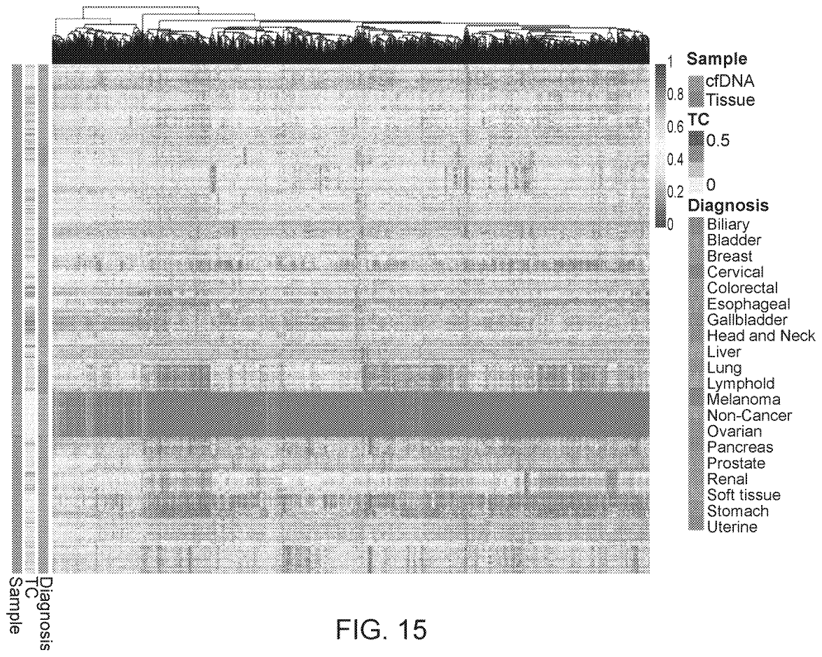

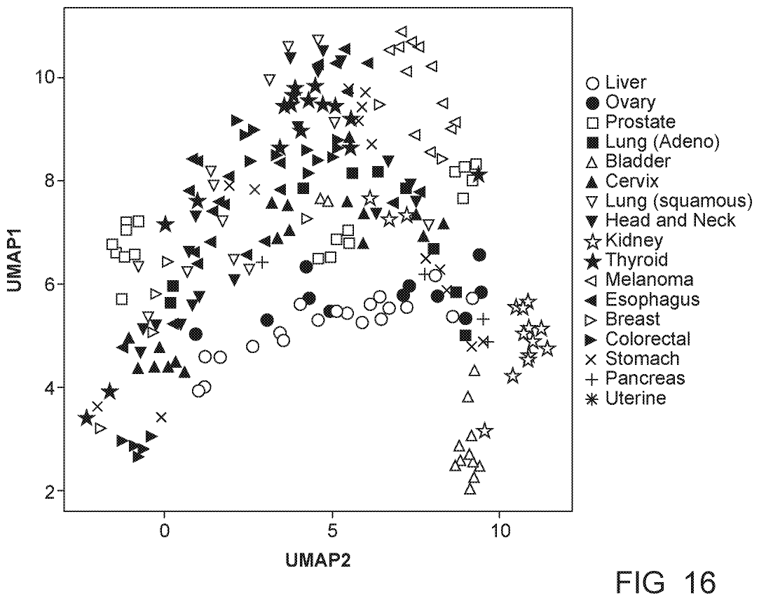

A first approach identified 130 continuous low methylation signal regions (low background regions; LBRs) in non-cancer cfDNA that had high signal in reference cancer tissue samples, as shown in FIG. 14. The LBRs are identified in Table 9. The second approach, used 448 cfDNA samples to assess methylation distibution in non-cancer and cancer samples, and identified 4000 short CpG motifs (subset of informative methylation biomarkers; SIMBA) that had higher methylation signals in non cancer cfDNA than in cancer samples, as shown in FIG. 15. The SIMBA biomarkers are identified in Table 8. Both biomarker sets showed low methylation in non-cancer cfDNA and high methylation in cancer tissue. Additionally, for both biomarker sets there was a lack of cancer-type specific methylation pattern. FIG. 16 shows a uniform manifold approximation and projeciton (UMAP) plot of SIMBA biomarkers and cancer samples, that supports a pan-cancer application.

The two biomarker sets (biomarkers of Tables 8 and 9) were combined to generate a 156 kb panel of informative biomarkers, which were further combined with optimized sequencing depth and hybrid capture conditions. Sequencing performance was evaluated under two hybrid-capture pooling conditions to assess the effect of higher multiplexing on data quality and efficiency, as shown in Table A. Increasing the hybrid capture pool plexity from 16 to 48 libraries per pool and flow cell capacity from 96 to 144 libraries per sequencing run, led to optimized assay efficiency, throughput and reduced cost by approximately 80% relative to a 18.6 Mb panel of 4,059 plus biomarkers.

TABLE A
Sequencing Performace for Hybrid Capture Conditions

| Sequencing metrics | 16-plex | 48-plex |
|---|---|---|
| On-target rate | 55.88% | 84.05% |
| Duplication rate | 57.74% | 80.44% |
| Ratio 90/10 | 2.105 | 2.120 |
| MTC (10ng input) | 111 | 143 |
| HS library size (10 ng input) | 279 K | 363 K |
| MTC (30 ng input) | 391 | 400 |
| HS library size (30 ng input) | 1,005 K | 1,022 K |

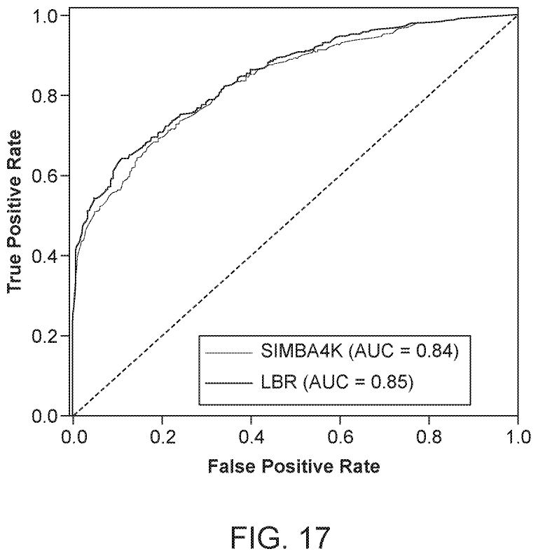

This combined model was used to process 1,342 cfDNA samples, that had 751 cancer samples with over 20 cancer types and 591 non-cancer samples. FIG. 17 shows the Area Under the Receiver Operating Characteristic (AUROC) curves for LBR and SIMBA (also referred to as SIMBA4K) biomarker sets, with LBR biomarkers showing slightly higher AUROC but overall similar performance for both biomarker sets on the 1,342 cfDNA samples.

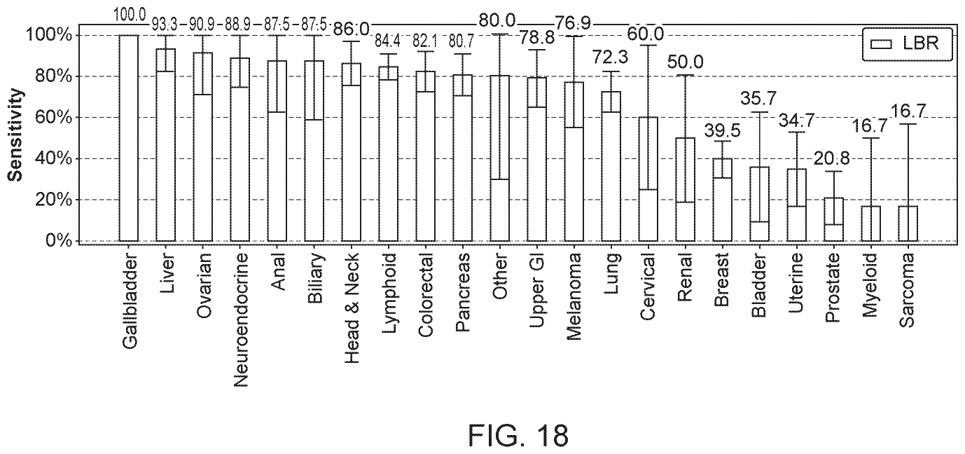

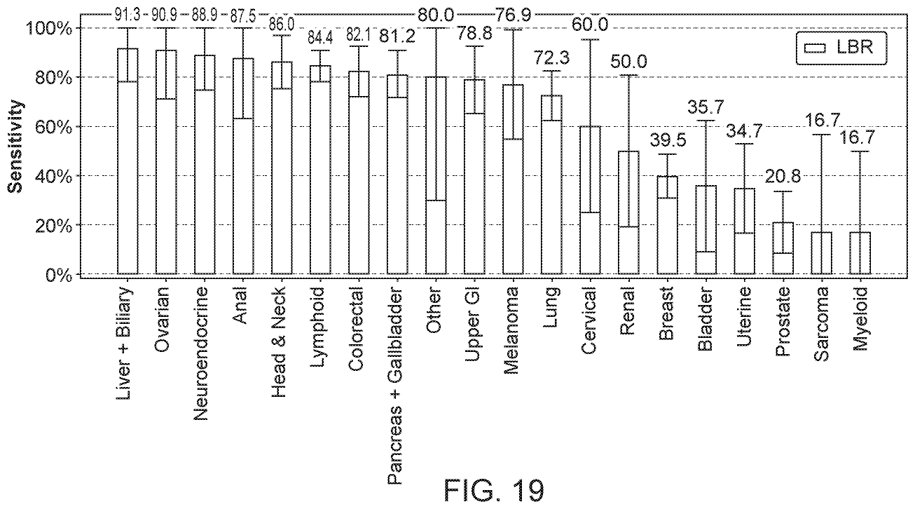

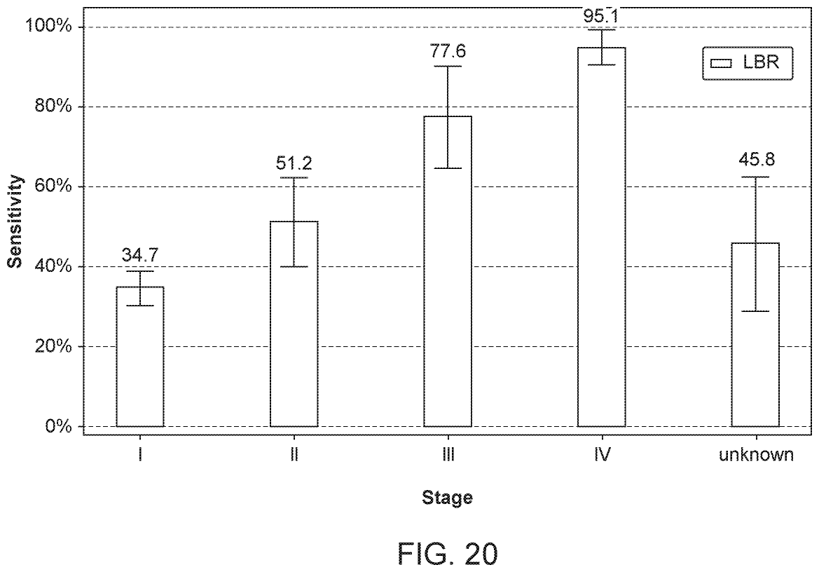

FIG. 18 and FIG. 19 show sensitivity by cancer type using LBR biomarkers. In a set of high mortality cancers without existing screening modalities, greater than 78% sensitivity was achieved at 80% target specificity. Overall, results were 62.7% sensitivity and 80.2% specificity. Furthermore, FIG. 20 shows cancer stage-specific sensitivity at 80% target specificity using LBR biomarkers, where higher sensitivity was observed in advanced cancer stages.

Independent binary prediction models were trained on the generated panel of biomarkers and evaluated via cross-validation to evaluate cancer detection performance.

Finally, a refined model for nine high-mortality cancer types was trained using additional cfDNA, with 218 cancer and 823 non-cancer samples, and synthetic data. This was validated in an independent blinded cohort with 466 cancer and 1062 non-cancer samples.

For a cohort of selected high-mortality cancers, at 80% target specificity, 80.3% overall sensitivity was observed, as shown in Table B. Additionally, at 82.8% specificity using a cancer detection model trained using LBR features, 48.5%, 76.5%, 92.5% and 94% sensitivity was observed for cancer stage I-IV, respectively, as shown in Table C.

TABLE B
Assay Sensitivity for High-Mortality Cancers

| Cancer type | Sensitivity | CI-low | CI-up |
|---|---|---|---|
| Lung (N = 242) | 76.4% | 70.7% | 81.4% |
| Upper GI (N = 71) | 78.9% | 67.8% | 86.9% |
| Pancreatobiliary (N = 68) | 85.3% | 74.7% | 91.9% |
| Head and Neck (N = 65) | 87.7% | 77.2% | 93.8% |
| Hepatobiliary(N = 20) | 90.0% | 66.8% | 97.6% |
| Overall | 80.3% | 76.4% | 83.6% |

TABLE C
Assay sensitivity for Cancer Stages

| Stage | Sensitivity | CI-low | CI-up |
|---|---|---|---|
| Stage I (N = 99) | 48.5% | 38.8% | 58.3% |
| Stage II (N = 68) | 76.5% | 64.9% | 85.1% |
| Stage III (N = 107) | 92.5% | 85.7% | 96.2% |
| Stage IV (N = 166) | 94.0% | 89.2% | 96.7% |

Without wishing to be bound by theory, the development of the 156 kb panel delivered robust performance across diverse cancer types demonstrating a cost-effective, scalable, and clinically informative assay with high sensitivity and NPV, that could augment current and future diagnostics to optimize clinical utility and throughput.

### Example 6: Example qMSP Biomarkers for Prioritizing Patient Care

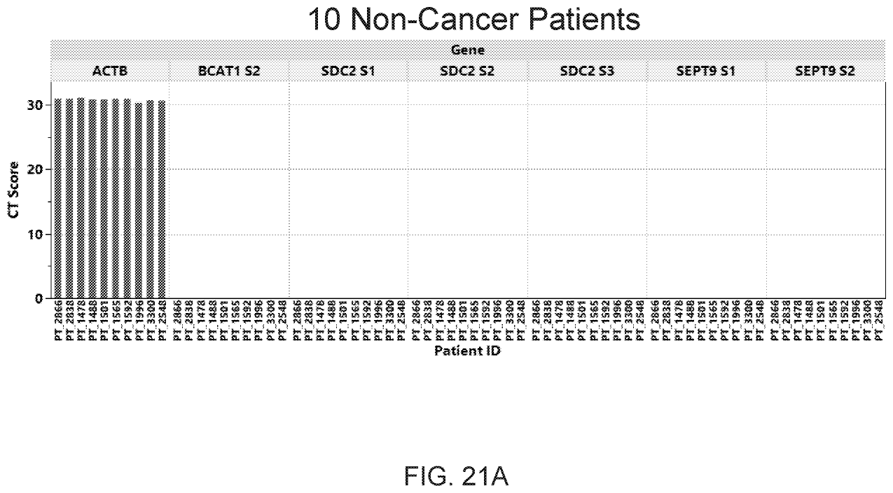

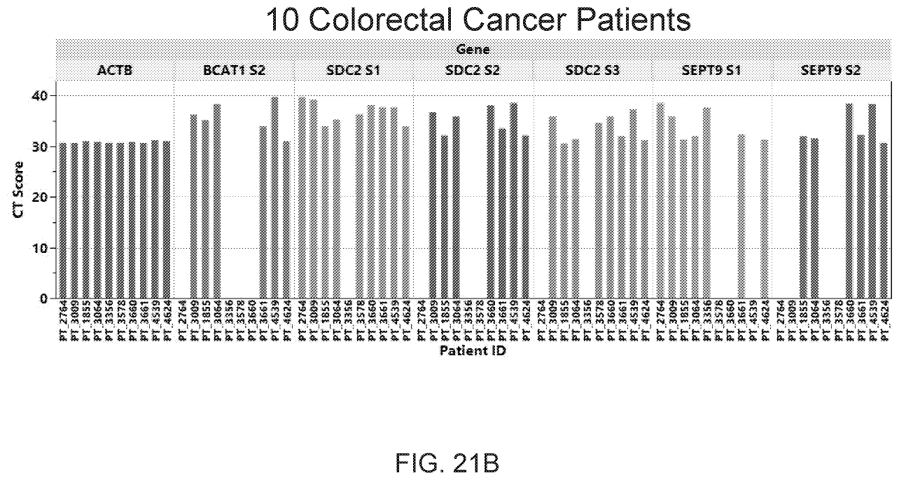

FIGS. 21A and 21B shows CT scores in 10 Cancer and 10 Non-Cancer Patients using qMSP biomarkers (BCAT1, SDC2, and SEPT9). ACTB is a control gene. Specifically, of the 20 patient samples, 10 were of low signal non-cancers, and 10 were of Stage 4 Colorectal Cancer Patients. Samples underwent cfDNA extraction and bisulfite conversion. BCAT1, Septin9, and SDC2 primers designed to target fully methylated CpG sites in target regions shown in the below table.

| Gene | Primer Set | Target Region (GRCh38/hg38) |
|---|---|---|
| BCAT1 | Set 1 (“S1”) | chr12c: 24,949,152 - 24,949,059 |
| | Set 2 (“S2”) | chr12: 24,948,928 - 24,949,056 |
| Septin9 | Set 1 (“S1”) | chr17: 77,373,473 - 77,373,555 |
| (“SEPT9”) | Set 2 (“S2”) | chr17c: 77,373,636 - 77,373,541 |
| SDC2 | Set 1 (“S1”) | chr8: 96,493,533 - 96,493,673 |
| | Set 2 (“S2”) | Ichr8: 96,494,094 - 96,494,187 |
| | Set 3 (“S3”) | chr8: 96,494,140 - 96,494,235 |

qPCR was then performed. FIG. 21A shows that no qMSP signal was detected in the non-cancer patients across all of target regions and corresponding primers. In contrast, FIG. 21B shows that the majority of the 10 CRC patients exhibited detectable qMSP signal across the various target regions using the corresponding primers. This emphasizes that the CT scores can be used to differentiate between cancer and non-cancer patients, thereby enabling stratification and prioritization of healthcare resources for such patients.

Lengthy table referenced here
US20260260740A1-20260903-T00001
Please refer to the end of the specification for access instructions.

Lengthy table referenced here
US20260260740A1-20260903-T00002
Please refer to the end of the specification for access instructions.

Lengthy table referenced here
US20260260740A1-20260903-T00003
Please refer to the end of the specification for access instructions.

Lengthy table referenced here
US20260260740A1-20260903-T00004
Please refer to the end of the specification for access instructions.

Lengthy table referenced here
US20260260740A1-20260903-T00005
Please refer to the end of the specification for access instructions.

Lengthy table referenced here
US20260260740A1-20260903-T00006
Please refer to the end of the specification for access instructions.

Lengthy table referenced here
US20260260740A1-20260903-T00007
Please refer to the end of the specification for access instructions.

Lengthy table referenced here
US20260260740A1-20260903-T00008
Please refer to the end of the specification for access instructions.

Lengthy table referenced here
US20260260740A1-20260903-T00009
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260260740A1). An electronic copy of the table will also be available from the USPTO upon request and payment of the fee set forth in 37 CFR 1.19(b)(3).

1-19. (canceled)

20. A method for prioritizing healthcare resources for patient care, the method comprising:
(a) obtaining methylation sequencing data of cfDNA from a next generation sequencing assay for a plurality of genomic regions comprising at least 1000 different CpG islands (CGIs), wherein differential methylation of the at least 1000 different CGIs were previously demonstrated to be associated with the initiation of or further development of an oncogenic state, the cfDNA obtained from one or more samples obtained from a subject previously identified as at risk of a cancer through a prior screen;
(b) analyzing, using a machine learning model, at least the sequencing data of the cfDNA from the one or more samples obtained from the subject to generate a risk score for the subject; and
(c) assigning the subject, based on the risk score, into a risk category selected from a plurality of stratified risk categories, each risk category of the plurality of risk categories having a corresponding frequency of healthcare utilization such that the recommended frequency of healthcare utilization of a subject in a lowest risk category is lower than the recommended frequency of healthcare utilization determined for a subject in other categories in the plurality of risk categories.

21. The method of claim 20, where the plurality of genomic regions are selected from genomic regions shown in any of Tables 4-9 or found in Septin9, SDC2, or BCAT1.

22. The method of claim 20, further comprising repeating steps (a)-(c) at one or more additional timepoints depending on the risk category of the subject.

23. The method of claim 20, wherein the plurality of stratified risk categories are generated by reference to at least two thresholds.

24. The method of claim 20, wherein the plurality of risk categories comprises a high risk category, an intermediate risk category, and a low risk category, wherein a frequency of healthcare utilization determined for the subject in the high risk category is greater than the frequency of healthcare utilization determined for a subject in the intermediate risk category and/or the low risk category.

25. The method of claim 20, wherein obtaining methylation sequencing data of cfDNA comprises processing bisulfite converted nucleic acids derived from cell-free DNA (cfDNA).

26. The method of claim 20, wherein obtaining methylation sequencing data comprises performing hybrid capture to enrich for the plurality of genomic regions selected from genomic regions shown in any of Tables 4-9 or found in Septin9, SDC2, or BCAT1.

27. The method of claim 20, wherein the healthcare utilization comprises a medical procedure, a future visit to a hospital, the use of a specialist, a diagnostic testing, a diagnostic imaging, a biopsy, a medical appointment, and/or a prophylactic medication.

28. The method of claim 20, wherein the prior screen comprises a family history evaluation, a life style evaluation, an age group, a demographic group, and/or a prior cancer diagnostic test.

29. The method of claim 28, wherein the prior screen comprises COLOGUARD® (Exact Sciences), CANCERGUARD™ (Exact Sciences), SHIELD™ (Guardant Health), Freenome Multiomic Blood test (Freenome), Everlywell fecal immunohistochemical test (FIT), Pinnacle Biolabsl FIT, iDNA HPV test, imaware Prostate Cancer screening test a PSA blood test, a mammogram, a colonoscopy, a Pap smear, an HPV test, a biopsy, a hereditary cancer screen (for example the Natera Empower test, Guardant Hereditary cancer test, Myriad Genetics hereditary cancer test or Labcorp hereditary cancer test), low-dose computed tomography (LDCT), a prenuvo cancer scan, or the Grail Galleri multi-cancer screening.

30. The method of claim 20, wherein the cancer is GI cancer selected from any of colorectal cancer, esophageal cancer, stomach cancer, gallbladder cancer, liver cancer, pancreatic cancer, small intestinal cancer, rectal cancer, appendix cancer, anal cancer, and/or colon cancer.

31. The method of claim 22, wherein the one or more additional timepoints comprises between 1 and 50 additional time points.

32. The method of claim 20, wherein the subject has a first-degree relative who was previously diagnosed with cancer prior to a threshold age of 45 years old, 50 years old, or 55 years old.

33. A method for assigning a healthcare utilization to a patient, the method comprising:
(a) obtaining methylation sequencing data of cfDNA from a next generation sequencing assay for a plurality of genomic regions, comprising at least 1000 different CpG islands (CGIs), wherein differential methylation of the at least 1000 different CGIs were previously demonstrated to be associated with the initiation of or further development of an oncogenic state, the cfDNA obtained from one or more samples obtained from a subject exhibiting symptoms indicative of cancer;
(b) analyzing, using a machine learning model, at least the sequencing data of the cfDNA from the one or more samples obtained from the subject to generate a risk score for the subject; and
(c) assigning the subject, based on the risk score, into a risk category selected from a plurality of stratified risk categories generated by reference to at least two thresholds, each risk category of the plurality of risk categories having a corresponding healthcare utilization such that the healthcare utilization of a subject in a low risk category is lower than the healthcare utilization determined for a subject in other categories in the plurality of risk categories.

34. The method of claim 33, where the plurality of genomic regions are selected from genomic regions shown in any of Tables 4-9 or found in Septin9, SDC2, or BCAT1.

35. The method of claim 33, further comprising repeating steps (a)-(c) at one or more additional timepoints depending on the risk category of the subject.

36. The method of claim 33, wherein the plurality of risk categories comprises a high risk category, an intermediate risk category, and a low risk category, wherein a healthcare utilization determined for the subject in the high risk category is greater than the healthcare utilization determined for a subject in the intermediate risk category and/or the low risk category.

37. The method of claim 33, wherein obtaining methylation sequencing data of cfDNA comprises processing bisulfite converted nucleic acids derived from cell-free DNA (cfDNA).

38. The method of claim 33, wherein the healthcare utilization comprises a medical procedure, a future visit to a hospital, the use of a specialist, a diagnostic testing, a diagnostic imaging, a biopsy, a medical appointment, and/or a prophylactic medication.
